(19) United States
(12) Patent Application Publication
(10) Pub. No.: US 2008/0069849 A1
(43) Pub. Date: Mar. 20, 2008

(54) Novel Antimicrobial Peptides

(75) Inventors: Artur Schmidtchen, Lund (SE); Martin Malmsten, Taby (SE); Bjorn Walse, Lund (SE)

Correspondence Address: MERCHANT & GOULD PC, P.O. BOX 2903, MINNEAPOLIS, MN, 55402-0903, US

(73) Assignee: Dermagen AB, Lund (SE)

(21) Appl. No.: 11/718,861

(22) PCT Filed: Nov. 17, 2005
(86) PCT No.: PCT/SE05/01737

§ 371 Date: Oct. 9, 2007

Related U.S. Application Data
(60) Provisional application No. 60/628,110, filed on Nov. 17, 2004.

(30) Foreign Application Priority Data
Nov. 17, 2004 (SE) 0402807-2

(51) Int. Cl. A61K 9/00; A01N 25/00; A01N 37/18; A01N 59/12; A01N 59/16; A01N 59/20; A01P 1/00; A61F 2/02; A61K 9/28; A61P 31/00; A61K 9/70; A61K 9/02; A61K 33/18; A61K 33/34; A61K 33/38; A61K 33/40; A61K 38/16; A61K 8/64

(52) U.S. Cl. 424/404; 424/422; 424/431; 424/445; 424/447; 424/474; 424/49; 424/59; 424/616; 424/618; 424/630; 424/667; 514/12; 514/13; 514/8

(57) ABSTRACT

The invention relates to the use of peptides, wherein at least one amino acid residue has been substituted to improve the efficacy of the antimicrobial peptide for the manufacturing of an antimicrobial composition. The composition can be used as a pharmaceutical composition to combat microorganisms, such as bacteria, virus, fungus, parasites as well as yeast.

### FIELD OF INVENTION

The invention relates to the use of peptides comprising the SEQ ID NO:1, wherein at least one amino acid residue has been substituted to improve the efficacy of the peptide for the manufacturing of an antimicrobial composition. The composition can be used as a pharmaceutical composition to combat microorganisms, such as bacteria, viruses, fungi, including yeast and parasites.

### BACKGROUND OF INVENTION

Several infections are successfully combated by the immune system of a mammal such as a human being. However, in some instances, bacteria, fungi, or viruses are not always cleared, which may cause localized or generalised acute infections. This is a serious concern at perinatal-, burn, or intensive care units, and in immunocompromised individuals. In other cases, a continuous bacterial persistence at epithelial surfaces may cause or aggravate chronic disease. In humans, this is exemplified by chronic skin ulcers, atopic dermatitis and other types of eczema, acne, or genitourinary infections.

Symptomatic infections may be treated by various medicaments. Some diseases may also be combated by for instance vaccines. However, vaccines are not always the best treatment option and for certain microorganisms no vaccine is available. When no protection is available treatment of the disease is pursued. Often the treatment is performed by the use of an antibiotic agent, which kills the microbe. However, during the last years several microbes have become resistant against anti-biotic agents. Most likely, resistance problems will increase in the near future. Additionally, several individuals have developed allergy against the antibiotic agent, thereby reducing the possibility to effectively use certain antibiotic agents.

Epithelial surfaces of various organisms are continuously exposed to bacteria. During recent years the innate immune system, based on antibacterial peptides has been attributed important roles in the initial clearance of bacteria at biological boundaries susceptible to infection (Lehrer, R. I., and Ganz, T. (1999) Curr Opin Immunol 11: 23-27, Boman, H. G. (2000) Immunol. Rev. 173, 5-16). Antimicrobial peptides kill bacteria by permeating their membranes, and thus the lack of a specific molecular microbial target minimizes resistance development.

Several antimicrobial peptides and proteins, unrelated to the herein, described peptides are known in the art.

U.S. Pat. No. 6,503,881 discloses cationic peptides being an indolicidin analogue to be used as an antimicrobial peptide. The cationic peptides being derived from different species, including animals and plants.

U.S. Pat. No. 5,912,230 discloses anti-fungal and anti-bacterial histatin-based peptides. The peptides being based on defined portions of the amino acid sequences of naturally occurring human histatins and methods for treatment of fungal and bacterial infections.

U.S. Pat. No. 5,717,064 discloses methylated lysine-rich lytic peptides. The lytic peptides being tryptic digestion resistant and non-natural. The lytic peptides are suitable for in vivo administration.

U.S. Pat. No. 5,646,014 discloses an antimicrobial peptide. The peptide was isolated from an antimicrobial fraction from silkworm hemolymph. The peptide exhibits excellent antimicrobial activity against several bacterial strains, such as Escherichia coli, Staphylococcus aureus and Bacillus cereus.

McCabe et al., J. Biol. Chem. Vol 277:27477-27488, 2002, describes an 37 kDa antimicrobial and chemotactic protein, azurocidin, containing the heparin binding consensus motifs XBBXBX and XBBBXXBX.

WO2004016653 discloses a peptide based on the 20-44 sequence of azurocidin. This peptide contains a loop structure linked by disulfide bridges.

U.S. Pat. No. 6,495,516 and related patents, disclose peptides based on the bactericidal 55 kDa protein bactericidal/permeability increasing protein (BPI). The peptides exerted antimicrobial effects as well as had heparin and LPS-neutralizing capacity.

WO 01/81578 discloses numerous sequences encoding G-coupled protein-receptor related polypeptides, which may be used for numerous diseases.

At present, over 700 different antimicrobial peptide sequences are known (www.bbcm.univ.trieste.it/˜tossi/search.htm), including cecropins, defensins magainins and cathelicidins.

Even though there is a relatively large number of antimicrobial peptides available today there is still an increased need of new improved antimicrobial peptides. Antimicrobial peptides, which can be used to combat microbes, microbes which are resistant or tolerant against antibiotic agents and/or other antimicrobial agents. More importantly, there is a need for new antimicrobial peptides, which are non-allergenic when introduced into mammals such as human beings. Bacteria have encountered endogenously produced antimicrobial peptides during evolution without induction of significant resistance.

### SUMMARY OF THE INVENTION

The invention relates to the use of new improved peptides comprising SEQ ID NO:1 and analogues thereof, wherein the peptide differs from SEQ ID NO:1 in that at least one amino acid residue selected from the group consisting C1, N2, T5, E6, R8, R9, H11, A12, R13, A14, S15, H16, L17, G18 and A20 has been substituted for the manufacturing of an antimicrobial composition to be used to combat microorganisms.

Additionally the invention relates to pharmaceutical compositions comprising the peptide and a pharmaceutically acceptable buffer, diluent, carrier, adjuvant or excipient.

Accordingly the invention relates to the use of a polypeptide which shows at least 70% homology to SEQ ID NO:2 for the manufacturing of an antimicrobial composition to prevent, inhibit, reduce or destroy microorganisms selected from the group consisting of bacteria, virus, parasites, fungus and yeast.

Finally the invention relates to a method of treating a mammal having a microbial infection, comprising administering to a mammal a therapeutically effective amount of an pharmaceutical composition comprising peptide and or peptides of the invention.

By providing such antimicrobial peptides, the risks for allergic reactions to antimicrobial peptides may be reduced due to the fact that the peptides are derived from the polypeptide sequence of endogenous proteins and/or peptides. By using short peptides the stability of the peptide is be increased and the production costs reduced, as compared to longer peptides and proteins, whereby the invention may be economically advantageous.

The peptides of the invention provide compositions, which facilitate efficient prevention, reduction or elimination of microorganisms. Thereby the possibility to combat microorganisms, which are resistant or tolerant against the antibiotic agents, may be increased. Moreover, mammals, which are allergic against commercially available antimicrobial agents, may be treated. By providing antimicrobial/pharmaceutical compositions, which are derived from endogenous improved proteins, the probability may be reduced or even eliminated that a mammal will develop allergy against these particular peptides. This makes the antimicrobial/pharmaceutical compositions useful for several applications in which the antimicrobial/pharmaceutical compositions contact a mammal either as a medicament or as an additive to prevent infections.

Additionally, the use of short peptides may improve bioavailability. Furthermore, the use of structurally distinct peptides with specific or preferable actions on Gram-negative and Gram-positive bacteria, or fungi, enables specific targeting of various microorganisms, thus minimising development of resistance and ecological problems. By using supplementing peptides, which are comparable to peptides already existing in the mammal, the risk of additional ecological pressure by novel antibiotics is further diminished. Finally, these formulations may also enhance the effect of endogenous antimicrobial peptides.

The inventive antimicrobial peptides increase the list of antimicrobial agents, which aid in the choice to prevent, reduce or eliminate microorganisms in all kind of applications including but not limited to those that invade or infect mammals, such as the human being.

### BRIEF DESCRIPTION OF THE DRAWINGS

FIG. 1 A describes bactericidal effects of CNY21 on E. faecalis 2374 (-●-), and P. aeruginosa 27.1 (-□-).

FIG. 1 B describes viable count analysis of CNY21 in different buffers.

FIG. 2 Helical wheel projection of the CNY21 peptide.

FIG. 3 Helical wheel projection of the CNYIEELRRQLLRALLRGLAR peptide.

FIG. 4a-c Plots of net charge as a function of RDA values for the different microorganisms Escherichia coli 37.4, Staphylococcus aureus isolate BD14312, Staphylococcus aureus ATCC29213, Candida albicans and hemolytic activity.

FIG. 5a-b Helical wheel projections of peptides 39, 42, 43 and 47.

FIG. 6 Schematic representation of an ideal amphipathic α-helix. CNY20 amino acid positions are represented by numbers in the helical wheel diagram. Black colour represents hydrophobic residues, white represents hydrophilic residues and gray represents the N- and C-terminus.

FIG. 7a-b Helical wheel projections of peptides with break of amphipathicity in the N-terminus, C-terminus, or central region.

FIG. 8. Describes radial diffusion assay analysis of CNY variants.

FIG. 9. Demonstrates antifungal effects of CNY-variants.

FIG. 10. Shows hemolytic effects of antimicrobial peptides.

FIG. 11. Illustrates effects of CNY-variants on eukaryotic membranes.

### DETAILED DESCRIPTION OF THE INVENTION

### Definitions

In the context of the present application and invention the following definitions apply:

The term “nucleotide sequence” is intended to mean a sequence of two or more nucleotides. The nucleotides may be of genomic DNA, cDNA, RNA, semisynthetic or synthetic origin or a mixture thereof. The term includes single and double stranded forms of DNA or RNA.

The term “substituted” is intended to mean that an amino acid residue is replaced by another amino acid residue. For example, S15V means that the serine amino acid residue in position number 15 in SEQ ID NO:1 has been substituted, i.e., replaced by valine.

The term “analogues thereof” is intended to mean that part of or the entire polypeptide of SEQ ID NO 1 is based on non protein amino acid residues, such as aminoisobutyric acid (Aib), norvaline gamma-aminobutyric acid (Abu) or ornithine. Examples of other non protein amino acid residues can be found at http://www.hort.purdue.edu/rhodcv/hort640c/polyam/po00008.htm.

The term “removed” is intended to mean that at least one amino acid residue has been removed, i.e., released from the polypeptide without being replaced by another amino acid residue.

The term “homology” is intended to mean the overall homology of the polypeptide SEQ ID N:2, not to be mixed up with the word “similarities” meaning that specific amino acid residues belong to the same group (i.e. hydrophobic, hydrophilic), or “identity”, meaning that amino acid residues are identical.

The term “antimicrobial peptide” is intended to mean a peptide, which prevents, inhibits, reduces or destroys a microorganism. The antimicrobial activity can be determined by any method, such as the method in EXAMPLE 3-5.

The term “amphipathic” is intended to mean the distribution of hydrophilic and hydrophobic amino acid residues along opposing faces of an α-helix structure, β-strand, linear, circular, or other secondary conformation, as well as along opposing ends of the peptide primary structure, which result in one face or end of the molecule being predominantly charged and hydrophilic and the other face or end being predominantly hydrophobic. The degree of amphipathicity of a peptide can be assessed, e.g., by plotting the sequence of amino acid residues by various web-based algorithms, e.g. those found on http://us.expasy.org/cgi-bin/protscale.pl or http://www.mbio.ncsu.edu/BioEdit/bioedit.html. The distribution of hydrophobic residues can be visualized by helical wheel diagrams. Secondary structure prediction algorithms, such as GORIV and AGADIR can be found at www.expasy.com.

The term “cationic” is intended to mean a molecule, which has a net positive charge within the pH range of from about 4 to about 12, such as within the range from about 4 to about 10.

The term “microorganism” is intended to mean any living microorganism. Examples of microorganisms are bacteria, fungus, virus, parasites and yeasts.

The term “antimicrobial agent” is intended to mean any agent, which prevent, inhibit or destroy life of microbes. Examples of antimicrobial agents can be found in The Sanford Guide to Antimicrobial Therapy (32nd edition, Antimicrobial Therapy, Inc, US).

In the present context, amino acid names and atom names are used as defined by the Protein DataBank (PDB) (www.pdb.org), which is based on the IUPAC nomenclature (IUPAC Nomenclature and Symbolism for Amino Acids and Peptides (residue names, atom names etc.), Eur J. Biochem., 138, 9-37 (1984) together with their corrections in Eur J. Biochem., 152, 1 (1985). The term “amino acid” is intended to indicate an amino acid from the group consisting of alanine (Ala or A), cysteine (Cys or C), aspartic acid (Asp or D), glutamic acid (Glu or E), phenylalanine (Phe or F), glycine (Gly or G), histidine (H is or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W) and tyrosine (Tyr or Y), or derivatives thereof.

### DESCRIPTION

Antimicrobial Peptide

The present invention relates to the use of peptides comprising SEQ ID NO:1 and analogues thereof, wherein the peptide differs from SEQ ID NO:1 in that at least one amino acid residue selected from the group consisting C1, N2, T5, E6, R8, R9, H11, A12, R13, A14, S15, H16, L17, G18 and A20 has been substituted for the manufacturing of an antimicrobial composition for the reduction or elimination of microorganisms. Substitutions which renders the polypeptide more active as compared to the peptide of SEQ ID NO:1. By utilising an algorithm based on helix-coil transition theory, AGADIR to predict helical properties suitable substitutions was identified (see Example 1). These substitutions increase the activity of the polypeptide in respect of the ability to combat microorganisms, since it appears that the antimicrobial potency is related to the inducibility of an α-helical conformation in a membrane-mimicking environment, rather than intrinsic helical stability (Tossi A, Sandri L, Giangaspero A. (2000), Amphipathic, alpha-helical antimicrobial, Biopolymers, 55, 4-30).

The substitution may be a change to another amino acid residue as well as to a non protein amino acid residue as long as the antimicrobial function of the polypeptide remains and/or is increased compared to the antimicrobial activity of SEQ ID NO:1.

The substitution(s) may be selected from the group consisting of C1G, N2S, N2T, N2K, T5E, T5D, T5N, E6A, E6V, E6L, E6I, E6M, E6F, E6Y, E6W, R8A, R8V, R8L, R8I, R8M, R8W, R8K, R9K, H11A, H11V, H11L, H11I, H11M, H11K, H11R, H11W, A12L, R13K, A14V, A14L, A14I, A14M, S15A, S15V, S15L, S15I, S15M, S15T, S15N, S15Q, S15K, S15R, S15W, H16K, H16R, H16A, H16V, H16L, H16I, H16M, L17K, L17R, L17A, L17V, L17I, L17M, G18W and A20R, such as selected from the group consisting of N2S, N2K, T5E, T5D, T5N, E6A, E6V, E6L, E6I, E6M, E6F, E6Y, E6W, R8A, R8V, R8L, R8I, R8M, R8K, R8W, H11A, H11V, H11L, H11I, H11M, H11K, H11R, H11W, A12L, A14L, S15A, S15V, S15L, S15I, S15M, S15T, S15N, S15Q, S15K, S15R, S15W, H16K, H16R, H16A, H16V, H16L, H16I, H16M, L17K, L17R, L17A, L17V, L17I, L17M, G18W and A20R.

Additionally, the invented antimicrobial peptide(s) may differ in 2, 3, 4, 5, 6, 7, 8, 9, 10, 11 or 12 amino acid residues from the amino acid sequence shown in SEQ ID NO:1.

Accordingly, one or more amino acid residue(s) may be removed from SEQ ID NO:1, both at C and N terminal part as well as from one of the parts as long as the antimicrobial activity remains. Example of amino acid resides that may be removed from SEQ ID NO:1 are C1, N2, T5, E6, R8, R9, H11, A12, R13, A14, S15, H16, L17, G18 and A20. However, any of the above mentioned amino acid residues, which may be substituted may in principle also be removed. 1, 2, 3, 4, 5, 6, 7, 8, 9, 10 or 11 amino acid residues may be removed from the SEQ ID NO:1.

The peptides may have a size of 10, 11, 12, 13, 14, 15, 16, 17, 18, 19, 20 or 21 amino acid residues.

The above mentioned SEQ ID NO:1 may be originally based on part of the complement factor C3 molecule (see SEQ ID NO:2). However, it may be synthetic or even semisynthetic.

The antimicrobial peptides may be extended by one or more amino acid residues, such as 1-100 amino acid residues, 5-50 amino acid residues or 6, 7, 8, 9, 10, 11, 12, 13, 14, 15, 16, 17, 18, 19, 20, 21, 22, 23, 24, 25, 26, 27, 28, 29 or 30 amino acid residues. Such additional amino acids may duplicate a sequence contiguous to the sequence of the antimicrobial peptide derived from a non-antimicrobial protein. The number to be added depends on which microorganism to be combated in including, stability of the peptide, toxicity, the mammal to be treated or in which product the peptide should be in and which peptide structure the antimicrobial peptide is based upon. The number of amino acid residues to be added to the peptides depends also on the choice of production, e.g., expression vector and expression hosts and the choice of manufacturing the antimicrobial/pharmaceutical composition. The extension may be at the N- or C-terminal part or at both parts of the antimicrobial peptides as long as it does not disrupt the antimicrobial effect of the peptide. The antimicrobial peptides may also be a fusion protein, wherein the antimicrobial peptide is fused to another peptide.

Additionally the antimicrobial peptides may be operably linked to other known antimicrobial peptides or other substances, such other peptides, proteins, oligosaccharides, polysaccharides, other organic compounds, or inorganic substances. For example the antimicrobial peptides may be coupled to a substance which protect the antimicrobial peptides from being degraded within a mammal prior to the antimicrobial peptides has inhibited, prevented or destroyed the life of the microorganism.

Accordingly the antimicrobial peptides may be modified at the C-terminal part by amidation or esterification and at the N-terminal part by acylation, acetylation, PEGylation, alkylation and the like.

Examples of microorganism that are inhibited, prevented or destroyed by the antimicrobial peptide are bacteria, both Gram positive and Gram-negative bacteria such as Enterococcus faecalis, Escherichia coli, Pseudomonas aeruginosa, Proteus mirabilis, Streptococcus pneumoniae, Streptococcus pyogenes, Staphylococcus aureus, viruse, parasites, fungus and yeast, such Candida albicans and Candida parapsilosis.

The antimicrobial peptides may be obtained from a naturally occurring source, such as from a human cell, a c-DNA, genomic clone, chemically synthesised or obtained by recombinant DNA techniques as expression products from cellular sources.

The antimicrobial peptides may be synthesised by standard chemical methods, including synthesis by automated procedure. In general, peptide analogues are synthesised based on the standard solid-phase Fmoc protection strategy with HATU (N—[DIMETHYLAMINO-1H-1.2.3.-TRIAZOLO[4,5-B]PYRIDIN-1-YLMETHYLELE]-N-METHYLMETHANAMINIUM HEXAFLUOROPHOSPHATE N-OXIDE) as the coupling agent or other coupling agents such as HOAt-1-HYDROXY-7-AZABENZOTRIAZOLE. The peptide is cleaved from the solid-phase resin with trifluoroacetic acid containing appropriate scavengers, which also deprotects side chain functional groups. Crude peptide is further purified using preparative reversed-phase chromatography. Other purification methods, such as partition chromatography, gel filtration, gel electrophoresis, or ion-exchange chromatography may be used. Other synthesis techniques, known in the art, such as the tBoc protection strategy, or use of different coupling reagents or the like can be employed to produce equivalent peptides.

Peptides may alternatively be synthesised by recombinant production (see e.g., U.S. Pat. No. 5,593,866). A variety of host systems are suitable for production of the peptide analogues, including bacteria, such as E. coli, yeast, such as Saccharomyces cerevisiae or pichia, insects, such as Sf9, and mammalian cells, such as CHO or COS-7. There are many expression vectors available to be used for each of the hosts and the invention is not limited to any of them as long as the vector and host is able to produce the antimicrobial peptide. Vectors and procedures for cloning and expression in E. Coli can be found in for example Sambrook et al. (Molecular Cloning.: A Laboratory Manual, Cold Spring Harbor Laboratory Press, Cold Spring Harbor, N.Y., 1987) and Ausubel et al. (Current Protocols in Molecular Biology, Greene Publishing Co., 1995).

Finally, the peptides may be purified from plasma, blood, various tissues or the like. The peptides may be endogenous, or generated after enzymatic or chemical digestion of the purified protein. For example, a heparin binding protein may be digested by trypsin and the resulting antibacterial peptides further isolated in larger scale.

A DNA sequence encoding the antimicrobial peptide is introduced into a suitable expression vector appropriate for the host. In preferred embodiments, the gene is cloned into a vector to create a fusion protein. To facilitate isolation of the peptide sequence, amino acids susceptible to chemical cleavage (e.g., CNBr) or enzymatic cleavage (e.g., V8 protease, trypsin) are used to bridge the peptide and fusion partner. For expression in E. coli, the fusion partner is preferably a normal intracellular protein that directs expression toward inclusion body formation. In such a case, following cleavage to release the final product, there is no requirement for renaturation of the peptide. In the present invention, the DNA cassette, comprising fusion partner and peptide gene, may be inserted into an expression vector. Preferably, the expression vector is a plasmid that contains an inducible or constitutive promoter to facilitate the efficient transcription of the inserted DNA sequence in the host.

The expression vector can be introduced into the host by conventional trans-formation techniques such as by calcium-mediated techniques, electroporation, or other methods well known to those skilled in the art.

The sequence encoding the antimicrobial peptide may be derived from a natural source such as a mammalian cell, an existing cDNA or genomic clone or synthesised. One method, which may be used, is amplification of the antimicrobial peptide by the aid of PCR using amplification primers which are derived from the 5′ and 3′ ends of the antimicrobial DNA template and typically incorporate restriction sites chosen with regard to the cloning site of the vector. If necessary, translational initiation and termination codons can be engineered into the primer sequences. The sequence encoding the antimicrobial peptide may be codon-optimised for facilitate expression in the particular host as long as the choice of the codons are made considering the final mammal to be treated. Thus, for example, if the antimicrobial peptide is expressed in bacteria, the codons are optimised for bacteria.

The expression vector may contain a promoter sequence, to facilitate expression of the introduced antimicrobial peptide. If necessary, regulatory sequences may also be included, such as one or more enhancers, ribosome binding site, transcription termination signal sequence, secretion signal sequence, origin of replication, selectable marker, and the like. The regulatory sequences are operably linked to each other to allow transcription and subsequent translation. If the antimicrobial peptide is to be expressed in bacteria, the regulatory sequences are those which are designed to e used within bacteria and such are well-known for a person skilled in the art. Suitable promoters, such as constitutive and inducible promoters, are widely available and includes promoters from T5, T7, T3, SP6 phages, and the trp, lpp, and lac operons.

If the vector containing the antimicrobial peptide is to be expressed within bacteria examples of origin are either those, which give rise to a high copy number or those which give rise to a low copy, for example f1-ori and col E1 ori.

Preferably, the plasmids include at least one selectable marker that is functional in the host, which allows transformed cells to be identified and/or selectively grown. Suitable selectable marker genes for bacterial hosts include the ampicillin resistance gene, chloramphenicol resistance gene, tetracycline resistance gene, kanamycin resistance gene and others known in the art.

Examples of plasmids for expression in bacteria include the pET expression vectors pET3a, pET 11a, pET 12a-c, and pET 15b (available from Novagen, Madison, Wis.). Low copy number vectors (e.g., pPD100) can be used for efficient overproduction of peptides deleterious to the E. coli host (Dersch et al., FEMS Microbiol. Lett. 123:19, 1994).

Examples of suitable hosts are bacteria, yeast, insects and mammal cells. However, often either bacteria such as E. coli is used.

The expressed antimicrobial peptide is isolated by conventional isolation techniques such as affinity, size exclusion, or ionic exchange chromatography, HPLC and the like. Different purification techniques can be found in A Biologist's Guide to Principles and Techniques of Practical Biochemistry (eds. Wilson and Golding, Edward Arnold, London, or in Current Protocols in Molecular Biology (John Wiley & Sons, Inc).

Accordingly, Human skin mast cells secrete histamine following stimulation with purified human C3a (300 nM to 600 uM range, Klubota Y. J. Dermatol. 1992 19:19-26). Simultaneous activation of human mast cells via aggregated IgG and C3a led to additive degranulation. These data support a mechanism by which MC may contribute to the inflammatory component in many inflammatory skin diseases. Thus, the herein disclosed antimicrobial peptides may act as inhibitors of mast cell activation and can function in concert with their antimicrobial effects as novel antiinflammatory molecules.

Additionally, the invention relates to pharmaceutical compositions comprising an antimicrobial peptide as described above and a pharmaceutical acceptable buffer, diluent, carrier, adjuvant or excipient. Additional compounds may be included in the compositions. These include, for example, chelating agents such as EDTA, EGTA or glutathione. The antimicrobial/pharmaceutical compositions may be prepared in a manner known in the art that is sufficiently storage stable and suitable for administration to humans and animals. The pharmaceutical compositions may be lyophilised, e.g., through freeze drying, spray drying or spray cooling.

“Pharmaceutically acceptable” means a non-toxic material that does not decrease the effectiveness of the biological activity of the active ingredients, i.e., the antimicrobial peptide(s). Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the peptide in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the peptide. The adjuvant may be one or more of zinc, copper or silver salts with different anions, for example, but not limited to fluoride, chloride, bromide, iodide, thiocyanate, sulfite, hydroxide, phosphate, carbonate, lactate, glycolate, citrate, borate, tartrate, and acetates of different acyl composition.

The excipient may be one or more of carbohydrates, polymers, lipids and minerals. Examples of carbohydrates include lactose, sucrose, mannitol, and cyclo-dextrines, which are added to the composition, e.g., for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The characteristics of the carrier are dependent on the route of administration. One route of administration is topical administration. For example, for topical administrations, a preferred carrier is an emulsified cream comprising the active peptide, but other common carriers such as certain petrolatum/mineral-based and vegetable-based ointments can be used, as well as polymer gels, liquid crystalline phases and microemulsions.

According to a specific embodiment the invention relates to an antimicrobial or pharmaceutical composition comprising a salt such as monovalent sodium, potassium, divalent zinc, magnesium, copper or calcium. The pH of that particular composition may be from about 4.5 to about 7.0, such as 5.0, 5.5, 6.0 or 6.5.

The compositions may comprise one or more peptides, such as 1, 2, 3 or 4 different peptides in the antimicrobial/pharmaceutical compositions. By using a combination of different peptides the antimicrobial effect may be increased and/or the possibility that the microorganism might be resistant and/or tolerant against the antimicrobial agent.

If the peptides are in a composition comprising a salt and/or a pH from about 4.5 to about 7.0 as defined above, the peptides become active, i.e., an enhanced effect is obtained by the addition of a salt and/or a choice of a specific pH range.

The peptide as a salt may be an acid adduct with inorganic acids, such as hydrochloric acid, sulfuric acid, nitric acid, hydrobromic acid, phosphoric acid, perchloric acid, thiocyanic acid, boric acid etc. or with organic acid such as formic acid, acetic acid, haloacetic acid, propionic acid, glycolic acid, citric acid, tartaric acid, succinic acid, gluconic acid, lactic acid, malonic acid, fumaric acid, anthranilic acid, benzoic acid, cinnamic acid, p-toluenesulfonic acid, naphthalenesulfonic acid, sulfanilic acid etc. Inorganic salts such as monovalent sodium, potassium or divalent zinc, magnesium, copper calcium, all with a corresponding anion, may be added to improve the biological activity of the antimicrobial composition.

The antimicrobial/pharmaceutical compositions of the invention may also be in the form of a liposome, in which the peptide is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfites, lysolecithin, phospholipids, saponin, bile acids, and the like. Preparation of such liposomal formulations is can be found in for example U.S. Pat. No. 4,235,871.

The antimicrobial/pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP0213303.

The antimicrobial/pharmaceutical compositions of the invention may also be in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the peptide.

Alternatively, the antimicrobial peptides may be dissolved in saline, water, polyethylene glycol, propylene glycol, ethanol or oils (such as safflower oil, corn oil, peanut oil, cottonseed oil or sesame oil), tragacanth gum, and/or various buffers. The pharmaceutical composition may also include ions and a defined pH for potentiation of action of antimicrobial peptides.

The antimicrobial/pharmaceutical compositions may be subjected to conventional pharmaceutical operations such as sterilization and/or may contain conventional adjuvants such as preservatives, stabilisers, wetting agents, emulsifiers, buffers, fillers, etc., e.g., as disclosed elsewhere herein.

The antimicrobial/pharmaceutical compositions according to the invention may be administered locally or systemically. Routes of administration include topical, ocular, nasal, pulmonary, buccal, parenteral (intravenous, subcutaneous, and intramuscular), oral, parenteral, vaginal and rectal. Also administration from implants is possible. Suitable antimicrobial preparation forms are, for example granules, powders, tablets, coated tablets, (micro) capsules, suppositories, syrups, emulsions, microemulsions, defined as optically isotropic thermodynamically stable systems consisting of water, oil and surfactant, liquid crystalline phases, defined as systems characterized by long-range order but short-range disorder (examples include lamellar, hexagonal and cubic phases, either water- or oil continuous), or their dispersed counterparts, gels, ointments, dispersions, suspensions, creams, aerosols, droplets or injectable solution in ampule form and also preparations with protracted release of active compounds, in whose preparation excipients, diluents, adjuvants or carriers are customarily used as described above. The pharmaceutical composition may also be provided in bandages, plasters or in sutures or the like.

The pharmaceutical compositions will be administered to a patient in a pharmaceutically effective dose. By “pharmaceutically effective dose” is meant a dose that is sufficient to produce the desired effects in relation to the condition for which it is administered. The exact dose is dependent on the, activity of the compound, manner of administration, nature and severity of the disorder, age and body weight of the patient different doses may be needed. The administration of the dose can be carried out both by single administration in the form of an individual dose unit or else several smaller dose units and also by multiple administration of subdivided doses at specific intervals

The pharmaceutical compositions of the invention may be administered alone or in combination with other therapeutic agents, such as antibiotic or antiseptic agents such as anti-bacterial agents, anti-fungicides, anti-viral agents, and anti-parasitic agents. Examples are penicillins, cephalosporins, carbacephems, cephamycins, carbapenems, monobactams, aminoglycosides, glycopeptides, quinolones, tetracyclines, macrolides, and fluoroquinolones. Antiseptic agents include iodine, silver, copper, chlorhexidine, polyhexanide and other biguanides, chitosan, acetic acid, and hydrogen peroxide. These agents may be incorporated as part of the same pharmaceutical composition or may be administered separately.

The present invention concerns both humans and other mammal such as horses, dogs, cats, cows, pigs, camels, among others. Thus the methods are applicable to both human therapy and veterinary applications. The objects, suitable for such a treatment may be identified by well-established hallmarks of an infection, such as fever, plus, culture of organisms, and the like. Infections that may be treated with the antimicrobial peptides include those caused by or due to microorganisms. Examples of microorganisms include bacteria (e.g., Gram-positive, Gram-negative), fungi, (e.g., yeast and molds), parasites (e.g., protozoans, nematodes, cestodes and trematodes), viruses, and pions. Specific organisms in these classes are well known (see for example, Davis et al., Microbiology, 3.sup.rd edition, Harper & Row, 1980). Infections include, but are not limited to, chronic skin ulcers, infected acute wounds and burn wounds, infected skin eczema, impetigo, atopic dermatitis, acne, external otitis, vaginal infections, seborrhoeic dermatitis, oral infections and parodontitis, candidal intertrigo, conjunctivitis and other eye infections, and pneumonia.

Accordingly, the antimicrobial/pharmaceutical compositions may be used for prophylactic treatment of burn wounds, after surgery and after skin trauma. The pharmaceutical composition may also be included in solutions intended for storage and treatment of external materials in contact with the human body, such as contact lenses, orthopedic implants, and catheters.

Additionally, the antimicrobial/pharmaceutical compositions may be used for treatment of atopic dermatitis, impetigo, chronic skin ulcers, infected acute wound and burn wounds, acne, external otitis, fungal infections, pneumonia, seborrhoeic dermatitis, candidal intertrigo, candidal vaginitis, oropharyngeal candidacies, eye infections (bacterial conjunctivitis), and nasal infections (including MRSA carriage).

The antimicrobial/pharmaceutical compositions may also be used to in cleansing solutions, such as lens disinfectants and storage solutions or used to prevent bacterial infection in association with urinary catheter use or use of central venous catheters.

Additionally, the antimicrobial compositions may be used for prevention of infection post-surgery in plasters, adhesives, sutures, or be incorporated in wound dressings.

The antimicrobial peptides may also be used in polymers, textiles or the like to create antibacterial surfaces or cosmetics, and personal care products (soap, shampoos, tooth paste, anti-acne, suncreams, tampons, diapers, etc) may be supplemented with the antimicrobial/pharmaceutical compositions.

The invention also relates to the use of a polypeptide which shows at least 70%, 80%, 90% or 95% or even more homology to SEQ ID NO:2, ie., the C3a polypeptide or the antimicrobial peptide as defined above or the antimicrobial/pharmaceutical composition as defined above for the manufacturing of an antimicrobial composition to prevent, inhibit, reduce or destroy microorganisms selected from the group consisting of bacteria, virus, parasites, fungus and yeast, as well as the use in therapy or diagnosis.

Finally, the invention relates to a method of treating a mammal having a microbial infection or suffering from allergy comprising administering to a patient a therapeutically effective amount of the pharmaceutical composition defined above.

### EXAMPLES

### Example 1

### Prediction of Potential Substitutions

An algorithm based on helix-coil transition theory, AGADIR, was used to predict helical propensity (Lacroix E, Viguera A R and Serrano L. (1998), Elucidating the folding problem of α-helices: local motifs, long-range electrostatics, ionic-strength dependence and prediction of NMR parameters, J Mol Biol, 284, 173-191). Calculations were performed by submitting peptide sequences to the EMBL WWW gateway to AGADIR service (http://www.emblheidelberg.de/Services/serrano/agadir/agadir-start.html).

Input parameters were as follow: Cterm free, Nterm free, pH 7.4, Temperature 278 K and Ionic Strength 0.15 M. Amphipathicity was investigated by generating helical wheel diagrams.

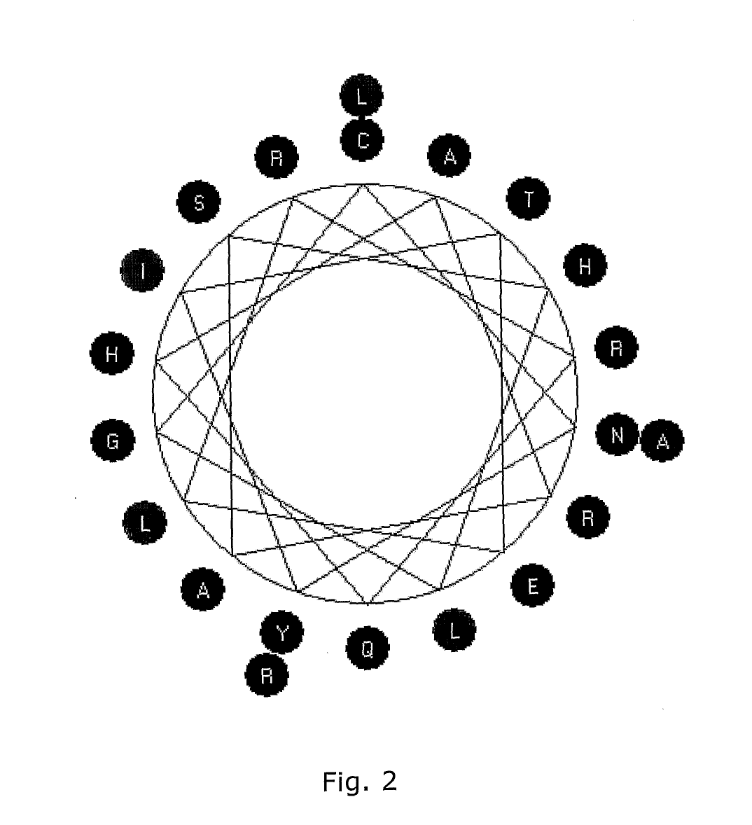

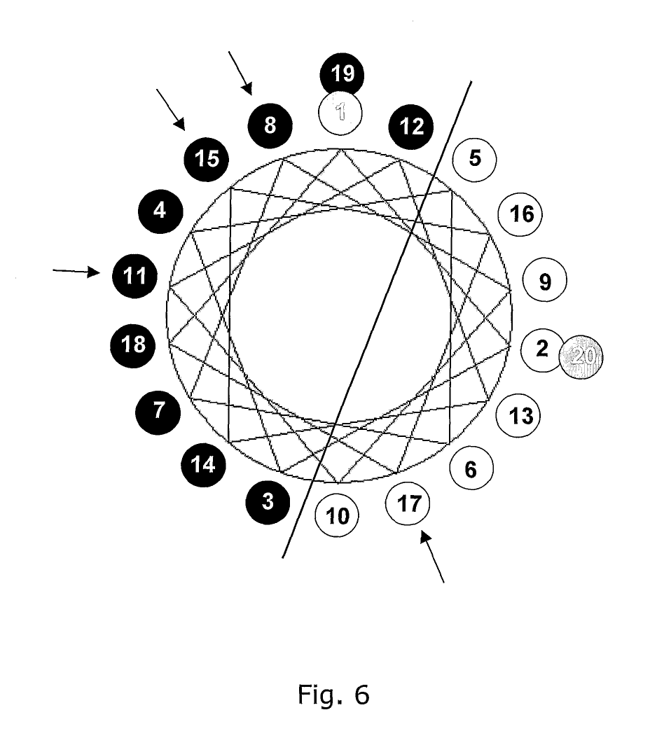

A structural analysis of CNY21 modelled to adopt an α-helical conformation shows that it has amphipathic character especially in the N-terminal part (FIGS. 2 and 6). In addition, the side-chains of Arg 9 and Gln 10 can form hydrogen bonds to the side-chain of Glu 6 and the side-chain of Arg 13 can form a hydrogen bond to the side-chain of Gin 10 stabilising the helical conformation.

It is well known that it exist amino acid preferences for specific locations at the ends of α-helices (Richardson J S and Richardson D C. (1988) Amino acid preferences for specific locations at the ends of α-helices, Science, 240, 1648-1652). The effects of different N-cap residues were investigated on CNY21 by AGADIR (the helical content predicted by AGADIR is shown after the peptide sequence).

| | |
|---|---|
| CNYITELRRQHARASHLGLAR | 4.83 |
| -NYITELRRQHARASHLGLAR | 13.58 |
| -SYITELRRQHARASHLGLAR | 14.57 |
| -GYITELRRQHARASHLGLAR | 4.85 |
| -TYITELRRQHARASHLGLAR | 4.47 |
| -VYITELRRQHARASHLGLAR | 3.16 |
| GNYITELRRQHARASHLGLAR | 4.97 |

As can be seen only removal of the C-terminal Cys and having either Asn or Ser as N-cap residues have a profound effect of the helical propensity. Furthermore, it has been shown that antimicrobial peptides has a positional conservation for Gly in the N-terminus (Tossi A, Sandri L, Giangaspero A. (2000), Amphipathic, alpha-helical antimicrobial peptides, Biopolymers, 55, 4-30). Here the replacement of the N-terminal Cys with Gly has little effect.

By assuming that Asn in position 2 is acting as a N-cap residue the investigation of different residues in the N-cap+3 position indicates a preference for Glu or Asp.

| | |
|---|---|
| CNYITELRRQHARASHLGLAR | 4.83 |
| CNYISELRRQHARASHLGLAR | 3.84 |
| CNYINELRRQHARASHLGLAR | 3.29 |
| CNYIQELRRQHARASHLGLAR | 2.03 |
| CNYIMELRRQHARASHLGLAR | 2.81 |
| CNYIDELRRQHARASHLGLAR | 11.64 |
| CNYIEELRRQHARASHLGLAR | 14.68 |
| CNYIGELRRQHARASHLGLAR | 2.42 |
| CNYIKELRRQHARASHLGLAR | 1.76 |
| CNYIRELRRQHARASHLGLAR | 2.08 |
| CNYIHELRRQHARASHLGLAR | 1.44 |
| CNYIYELRRQHARASHLGLAR | 2.87 |
| CNYIFELRRQHARASHLGLAR | 2.29 |
| CNYIWELRRQHARASHLGLAR | 3.15 |
| CNYIPELRRQHARASHLGLAR | 1.27 |

This preference is due to that a reciprocal backbone-side-chain hydrogen bond interaction is formed termed the capping box (Harper E T and Rose G D, (1993), Helix stop signals in proteins and peptides: The capping box, Biochemistry, 32, 7605-7609). Further analysis of this motif reveal a preference for either Asn, Ser or Thr in position 2 and a preference for Glu or Asp in position 5 of CNY21.

| | |
|---|---|
| CNYITELRRQHARASHLGLAR | 4.83 |
| CNYIEELRRQHARASHLGLAR | 14.68 |
| CNYIDELRRQHARASHLGLAR | 11.64 |
| CSYITELRRQHARASHLGLAR | 4.94 |
| CSYIEELRRQHARASHLGLAR | 18.89 |
| CSYIDELRRQHARASHLGLAR | 13.90 |
| CTYITELRRQHARASHLGLAR | 3.19 |
| CTYIEELRRQHARASHLGLAR | 14.81 |
| CTYIDELRRQHARASHLGLAR | 11.57 |

Also here the removal of the N-terminal Cys increase helicity especially for peptides with the NXXE and SXXE capping motif.

| | |
|---|---|
| -NYITELRRQHARASHLGLAR | 13.58 |
| -NYIEELRRQHARASHLGLAR | 27.33 |
| -NYIDELRRQHARASHLGLAR | 20.18 |
| -NYINELRRQHARASHLGLAR | 6.37 |
| -NYIHELRRQHARASHLGLAR | 3.26 |
| -NYIQELRRQHARASHLGLAR | 4.39 |
| -SYITELRRQHARASHLGLAR | 14.57 |
| -SYIEELRRQHARASHLGLAR | 32.01 |
| -SYIDELRRQHARASHLGLAR | 24.58 |
| -SYINELRRQHARASHLGLAR | 6.76 |
| -SYIHELRRQHARASHLGLAR | 3.50 |
| -SYIQELRRQHARASHLGLAR | 4.95 |
| -TYITELRRQHARASHLGLAR | 4.47 |
| -TYIEELRRQHARASHLGLAR | 22.89 |
| -TYIDELRRQHARASHLGLAR | 16.54 |
| -TYINELRRQHARASHLGLAR | 4.61 |
| -TYIHELRRQHARASHLGLAR | 2.21 |
| -TYIQELRRQHARASHLGLAR | 3.46 |

Sometimes helices are stabilized by a hydrophobic residue at position N-cap+4. This was investigated also with the aim to see if a negative charge could be eliminated.

| | |
|---|---|
| CNYITELRRQHARASHLGLAR | 4.83 |
| CNYITALRRQHARASHLGLAR | 4.51 |
| CNYIEELRRQHARASHLGLAR | 14.68 |
| CNYIEALRRQHARASHLGLAR | 15.57 |
| CNYIELLRRQHARASHLGLAR | 16.61 |
| -NYITELRRQHARASHLGLAR | 13.58 |
| -NYIEELRRQHARASHLGLAR | 27.33 |
| -NYIEALRRQHARASHLGLAR | 9.57 |
| -NYIELLRRQHARASHLGLAR | 7.31 |

Helices are usually terminated with a Gly as C-cap residue or with Pro in the C-cap+1 position (Richardson J S and Richardson D C. (1988) Amino acid preferences for specific locations at the ends of α-helices, Science, 240, 1648-1652). CNY21 has a Schellman motif (Prieto J and Serrano L, (1997), C-capping and helix stability: The Pro C-capping motif, J Mol Biol, 274, 276-288) in its C-terminus identified by the fingerprint Gly in position i, a hydrophobic residue in i−4 and i+1 and a polar or Ala residue at position i−2.

| | |
|---|---|
| CNYITELRRQHARASHLGLAR | 4.83 |
| CNYITELRRQHARLSHLGLAR | 5.02 |
| CNYITELRRQHARASALGLAR | 5.86 |
| CNYITELRRQHARLSALGLAR | 6.53 |

Furthermore, helix content can drastically be increased by optimising the spacing between hydrophobic residues in the peptide. A spacing of i, i+3 or i, i+4 especially between leucines are known to stabilize helices with the latter spacing giving the strongest interaction (Luo P, Baldwin R L. (2002) Origin of the different strengths of the (i, i+4) and (i, i+3) leucine pair interactions in helices, Biophys Chem. 96, 103-108). Tyr 3 makes a favourable i, i+4 interaction with Leu 7 in the N-terminus of CNY21. The helix content is increased from 5% to almost 50% in CNY21 by inserting leucines at positions 8, 11, 12 and 16 as can be seen below.

| | |
|---|---|
| CNYITELRRQHARASHLGLAR | 4.83 |
| CNYITELLRQHARASHLGLAR | 10.59 |
| CNYITELRRQLARASHLGLAR | 15.15 |
| CNYITELRRQHLRASHLGLAR | 5.09 |
| CNYITELRRQHARASLLGLAR | 5.91 |
| CNYITELLRQLARASHLGLAR | 31.64 |
| CNYITELLRQLLRASHLGLAR | 39.43 |
| CNYITELRRQLARASLLGLAR | 17.90 |
| CNYITELRRQLLRASLLGLAR | 22.22 |
| CNYITELLRQLARASLLGLAR | 35.71 |
| CNYITELLRQLLRASLLGLAR | 47.49 |

The amphipathic structure of CNY21 is not optimal (FIGS. 2 and 6). By replacing Arg 8, H is 11 and Ser 15 with a hydrophobic residue and replacing H is 16 and Leu 17 with a hydrophilic charged residue, such as a positively charged amino acid to increase the net positive charge of the peptide will optimise the amphipathic character of CNY21.

| | |
|---|---|
| CNYITELLRQHARASHLGLAR | 10.59 |
| CNYITELLRQLARASHLGLAR | 31.64 |
| CNYITELLRQLARALHLGLAR | 47.29 |
| CNYITELRRQHARASHKGLAR | 5.07 |
| CNYITELLRQLARALHKGLAR | 48.70 |
| CNYITELRRQHARASKLGLAR | 6.01 |
| CNYITELLRQHARASKLGLAR | 12.70 |
| CNYITELLRQLARASKLGLAR | 36.26 |
| CNYITELLRQLARASKKGLAR | 37.53 |
| CNYITELLRQLARASQKGLAR | 36.48 |
| CNYITELLRQLARASEKGLAR | 32.67 |
| CNYITELLRQLARALKKGLAR | 57.55 |
| CNYITELLRQLLRALKKGLAR | 64.98 |

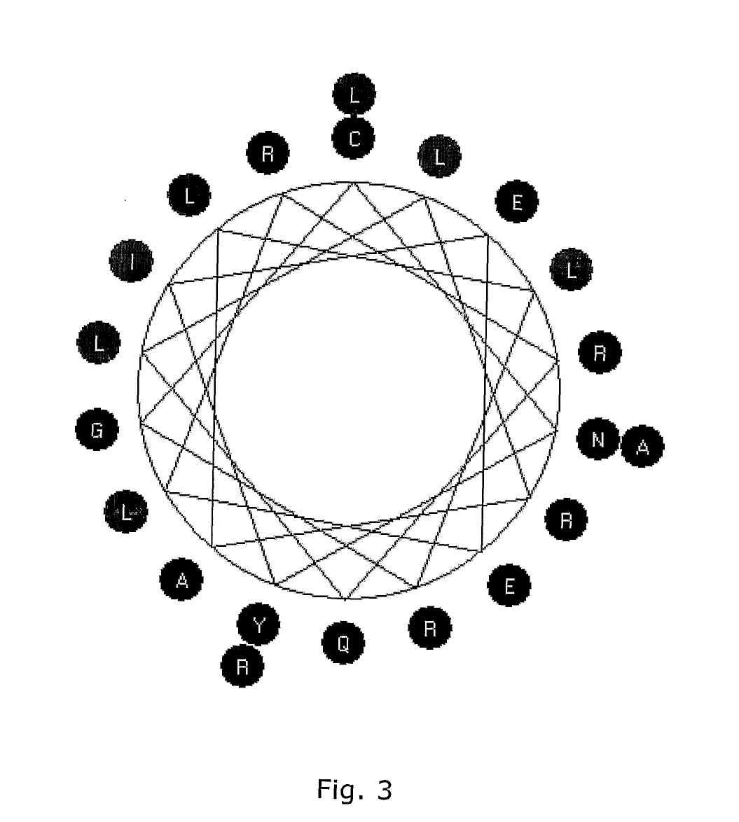

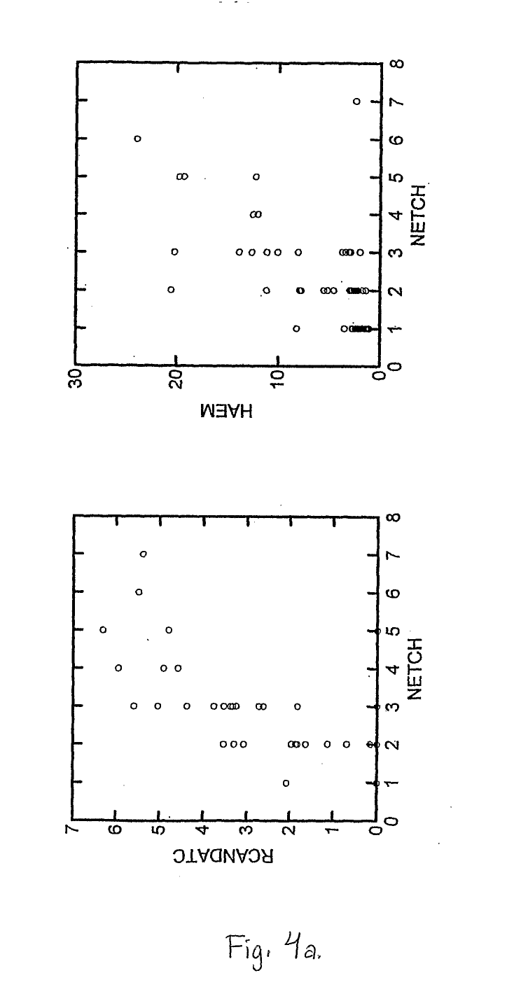

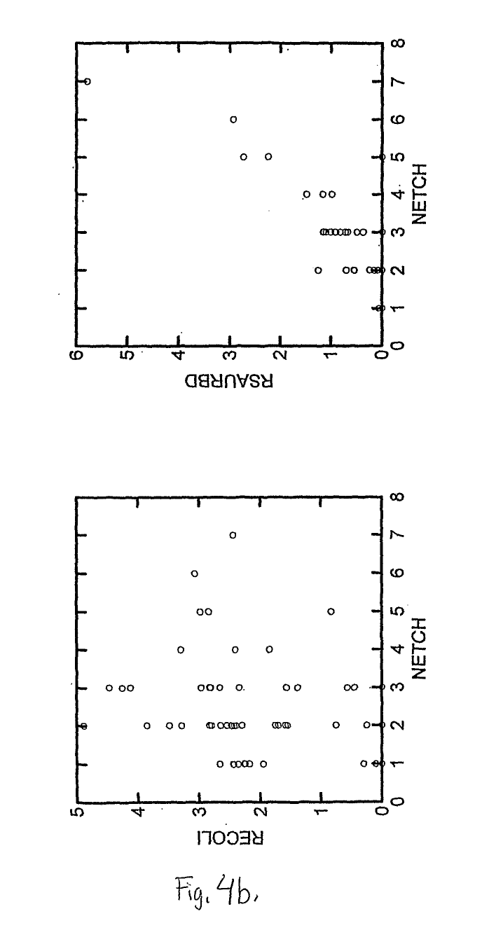

Finally, by combining the different substitutions described above it is possible to design variants of CNY21 with increased helicity and optimal amphipathicity. With only six substitutions it is possible to increase helicity from 5% to over 70% as exemplified by the peptide with substitutions T5E, H11L, A12L, S15L, H16L and L17R. A helical wheel projection of this CNY21 variant is shown in FIG. 3.

| | |
|---|---|
| CNYIEELLRQLARALHKGLAR | 58.71 |
| CSYIEELLRQLARALHKGLAR | 61.84 |
| CSYIEELLRQLLRALLKGLAR | 76.45 |
| CNYIEELRRQLARALHKGLAR | 51.32 |
| CNYIEELRRQLLRALHKGLAR | 57.97 |
| CSYIEELRRQLARALHKGLAR | 55.59 |
| CSYIEELRRQLLRALLKGLAR | 73.55 |
| CSYIEELRRQLLRALLRGLAR | 74.12 |
| CNYIEELRRQLLRALLKGLAR | 71.47 |
| CNYIEELRRQLLRALLRGLAR | 72.04 |
| CNYIEELLRQLLRALKKGLAR | 70.57 |
| CSYIEELLRQLLRALKKGLAR | 72.02 |
| CSYIEELLRQLLRALKRGLAR | 72.40 |

All proposed substitutions at various positions of the CNY21 peptide are summarized in Table 1.

TABLE 1

Amino acid substitutions of CNY21 that increase
helicity

```
1 2 3 4 5 6 7 8 9 1 1 1 1 1 1 1 1 1 1 2 2
                                    0 1 2 3 4 5 6 7 8 9 0 1
C N Y I T E L R R Q H A R A S H L G L A R
- S E A A K A L K V A K K
G T D V V V L V R R
                N L L L I L A A
                    I I I M I V V
                    M M M M L I
                    F I M
                    Y M
                    W
```

The negative control peptides CNY21R-S and CNY21H-P should have lesser helicity than CNY21. A much lower helical content is also correct predicted for these peptides. The peptides CNY21H-K and CNY21H-L displaying larger anti-bacterial effect have higher predicted helicity, which is in agreement with the hypothesis that the potency increases with larger propensity to adopt an α-helical conformation.

| | | |
|---|---|---|
| CNY21 | CNYITELRRQHARASHLGLAR | 4.83 |
| CNY21R-S | CNYITELSSQHASASHLGLAR | 0.60 |
| CNY21H-P | CNYITELRRQPARASPLGLAR | 1.91 |
| CNY21H-K | CNYITELRRQKARASKLGLAR | 9.72 |
| CNY21H-L | CNYITELRRQLARASLLGLAR | 17.90 |

### Example 2

### Antimicrobial Peptides

The peptides CNY21; CNYITELRRQHARASHLGLAR, CNY20; CNYITELRRQHARASHLGLA, CNY21R-S; CNYITELSSQHASASHLGLAR, CNY20R-S; CNYITELSSQHASASHLGLA, CNY21H-L: CNYITELRRQLARASLLGLAR, CNY21H-K: CNYITELRRQLARASKLGLAR, CNY21H-P: CNYITELRRQPARASPLGLAR were synthesised by Innovagen AB, Ideon, SE-22370, Lund, Sweden. The purity (>95%) and molecular weight of these peptides was confirmed by mass spectral analysis (MALDI.TOF Voyager).

Microorganisms

Enterococcus faecalis 2374, Escherichia coli 37.4, Pseudomonas aeruginosa 27.4, originally obtained from chronic venous ulcers, and the fungus Candida albicans BM 4435, obtained from a patient with atopic eczema, were used in the experiments.

### Example 3

### Antibacterial Effects of C3a-Derived CNY21 Peptide

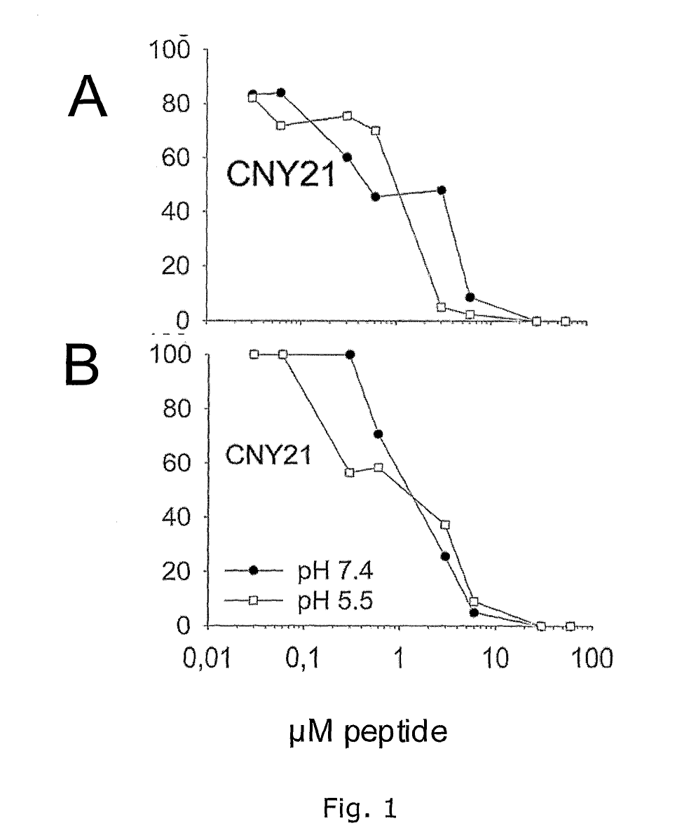

FIG. 1A describes bactericidal effects of CNY21 on E. faecalis 2374 (-●-), and P. aeruginosa 27.1 (-□-). Bacteria were grown to mid-logarithmic phase in Todd-Hewitt (TH) medium. Bacteria were washed and diluted in either 10 mM Tris, pH 7.4, containing 5 mM glucose Bacteria (50 μl; 2×10⁶ cfu/ml) were incubated, at 37° C. for 2 hours, with the synthetic peptide at concentrations ranging from 0.03 to 60 μM. To quantify the bactericidal activity, serial dilutions of the incubation mixture were plated on TH agar, followed by incubation at 37° C. overnight and the number of colony-forming units was determined.

FIG. 1 B describes viable count analysis of CNY21 in different buffers; 10 mM Tris pH 7.4 (-●-) and 10mM MES pH 5.5 (-□-), both containing 0.15 M NaCl. P. aeruginosa 27.1 (2×10⁶ cfu/ml) were incubated in 50 μl with peptides at concentrations ranging from 0.03 to 6 μM.

### Example 4

### Radial Diffusion Assay Analysis of CNY Variants

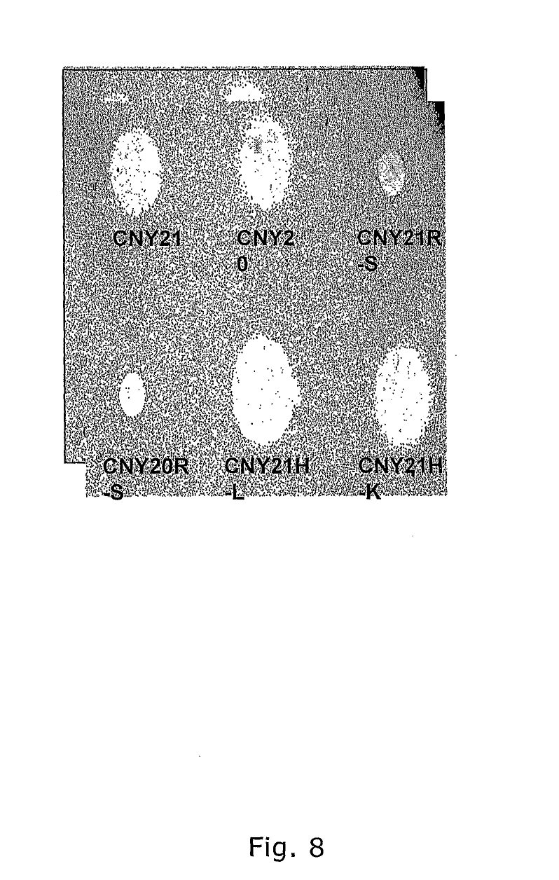

Radial diffusion assays (RDA) were performed essentially as described earlier (Andersson et al., Eur J Biochem, 2004, 271:1219-1226). Briefly, bacteria (P. aeruginosa 27.1) were grown to mid-logarithmic phase in 10 ml of full-strength (3% w/v) trypticase soy broth (TSB) (Becton-Dickinson, Cockeysville, Md.). The microorganisms were washed once with 10 nM: Tris, pH 7.4. 4×10⁶ bacterial cfu or 1×10⁵ fungal cfu was added to 5 ml of the underlay agarose gel, consisting of 0.03% (w/v) TSB, 1% (w/v) low-electroendosmosistype (Low-EEO) agarose (Sigma, St Louise Mo.) and a final concentration of 0.02% (v/v) Tween 20 (Sigma). The underlay was poured into a Ø 85 mm petri dish. After agarose solidified, 4 mm-diameter wells were punched and 6 μl of test sample was added to each well. Plates were incubated at 37° C. for 3 hours to allow diffusion of the peptides. The underlay gel was then covered with 5 ml of molten overlay (6% TSB and 1% Low-EEO agarose in dH₂O). Antimicrobial activity of a peptide is visualized as a clear zone around each well after 18-24 hours of incubation at 37° C. Synthetic peptides were tested in concentrations of 100 μM to determine the antibacterial effect (FIG. 8). CNY21; CNYITELRRQHARASHLGLAR, CNY20; CNYITELRRQHARASHLGLA, CNY21R-S; CNYITELSSQHASASHLGLAR, CNY20R-S; CNYITELSSQHASASHLGLA, CNY21H-L: CNYITELRRQLARASLLGLAR, CNY21H-K: CNYITELRRQKARASKLGLAR, CNY21H-P: CNYITELRRQPARASPLGLAR. The CNY211H-P variant (not shown here) exerted no antimicrobial effects.

### Example 5

### Antifungal Effects of CNY-Variants

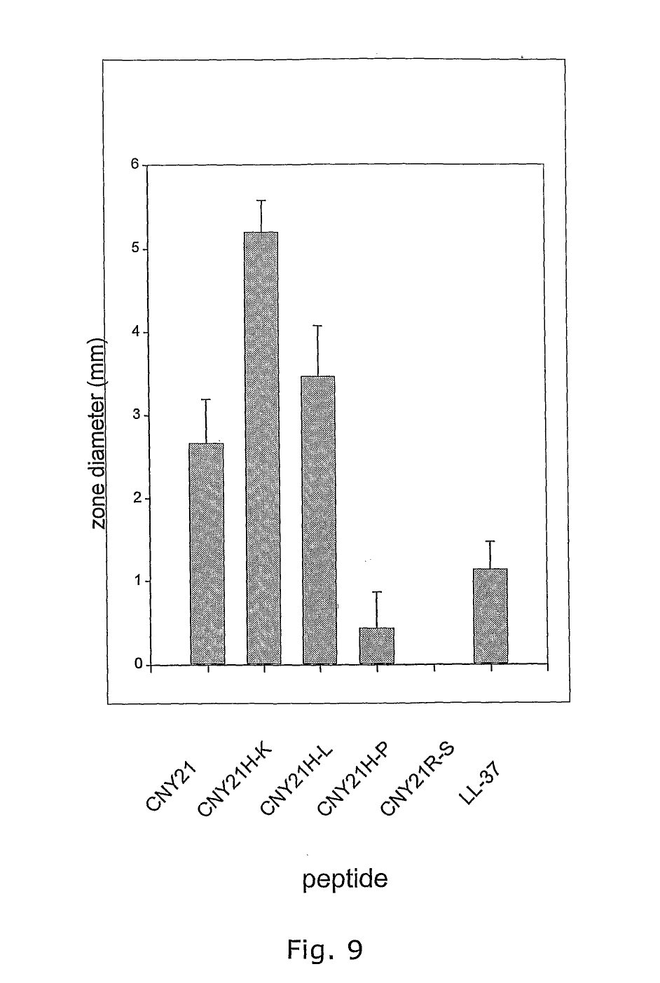

Fungi (C. albicans) were grown to mid-logarithmic phase in 10 ml of full-strength (3% w/v) trypticase soy broth (TSB) (Becton-Dickinson, Cockeysville, Md.). The microorganisms were washed once with 10 mM Tris, pH 7.4. 1×10⁵ fungal cfu was added to 5 ml of the underlay agarose gel, consisting of 0.03% (w/v) TSB, 1% (w/v) low-electroendosmosistype (Low-EEO) agarose (Sigma, St Louise Mo.) and a final concentration of 0.02% (v/v) Tween 20 (Sigma). The underlay was poured into a ø 85 mm petri dish. After agarose solidified, 4 mm-diameter wells were punched and 6 μl of test sample was added to each well. Plates were incubated at 37° C. for 3 hours to allow diffusion of the peptides. The underlay gel was then covered with 5 ml of molten overlay (6% TSB and 1% Low-EEO agarose in dH₂O). Antimicrobial activity of a peptide is visualized as a clear zone around each well after 18-24 hours of incubation at 28° C. for Candida albicans (FIG. 9). The results represent mean of triplicate samples. CNY21; CNYITELRRQHARASHLGLAR, CNY21H-K: CNYITELRRQKARASKLGLAR, CNY21H-L: CNYITELRRQLARASLLGLAR, CNY20R-S; CNYITELSSQHASASHLGLA, CNY21 R-S; CNYITELSSQHASASHLGLAR, CNY21H-P: CNYITELRRQPARASPLGLAR.

### Example 6

### Hemolytic Effects of Antimicrobial Peptides

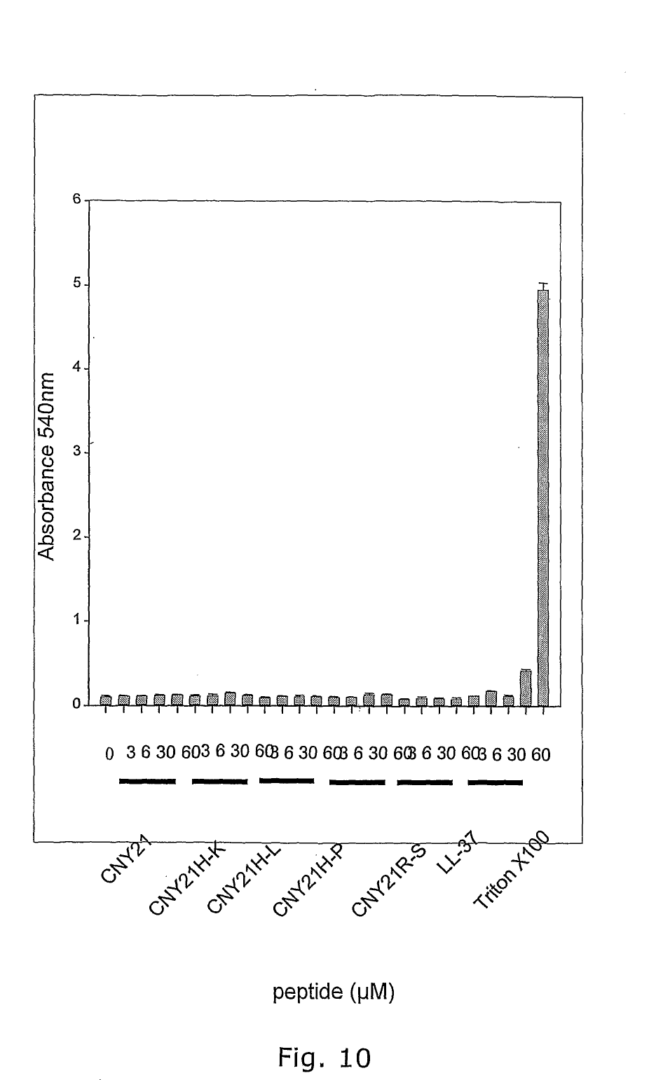

Hemolytic activity was determined by monitoring the release of hemoglobin at 540 nm. Briefly, a suspension of erythrocytes (10% in PBS) was incubated with an equal volume of peptide (in PBS). The mixture was incubated for 1 hour at 37 C and centrifuged. The absorbance of the supernatant was determined. One hundred percent hemolysis was reached by addition of an equal volume of 2% Triton X100 the erythrocyte suspension. The CNY variants studied exerted little or no hemolytic effects (FIG. 10). In comparison the antibacterial peptide LL-37 caused 6% hemolysis at 60 uM.

### Example 7

### Effects of CNY-Variants on Eukaryotic Membranes

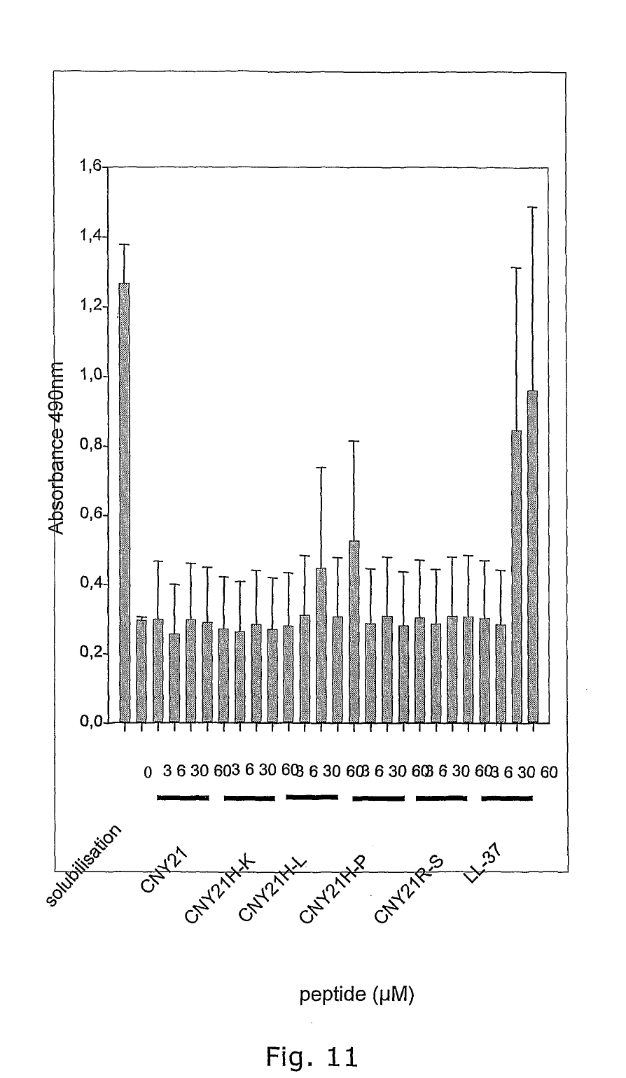

Membrane permeabilising effects towards the human HaCaT keratinocyte cell line were studied. Confluent cell cultures grown in 96-well plates were incubated with peptides for 6 hours, and the release of lactate dehydrogenase measured. The CNY variants studied did not release any LDH, in contrast to the antibacterial peptide LL-37 (FIG. 11).

### Example 8

### Heparin Binding of CNY21

Peptides were tested for heparin binding activities. Peptides were applied on nitrocellulose membranes (Hybond, Amersham Biosciences). Membranes were blocked (PBS, pH 7.4, 0.25% Tween 20, 3% bovine serum albumin) for one hour and incubated with radiolabelled heparin for one hour in the same buffer. Unlabelled polysaccharides (Heparin, 2 mg/ml) were added for competition of binding. The membranes were washed (3×10 min in PBS, pH 7.4, 0.25% Tween 20). A Bas 2000 radio imaging system (Fuji) was used for visualization of radioactivity. Unlabelled heparin (6 mg/ml) inhibited the binding of ¹²⁵I-heparin CNY21.

### Example 9

### Binding of CNY21 Variants to Lipid Bilayers

Peptides were tested for binding to lipid bilayers, resulting pore formation and peptide secondary structure in the lipid membranes. Lipid membranes investigated included both zwitterionic ones (containing phosphatidylcholine) and anionic ones (containing mixtures of phosphatidylcholine and phosphatidic acid). Lipid membranes were deposited at silica, and the binding of the peptides from 10 mM Tris, pH 7.4, was directly monitored by ellipsometry. Lipid membranes were also prepared in the form of liposomes from the same lipids and in the same buffer by extradition and repeated freeze-thawing, which resulted in unilamellar liposomes of 150 nm diameter. Pore formation in these liposomes were determined by including carboxyfluorescein in the liposomes and following the fluorescence intensity increase on addition of peptides to the liposomes. Furthermore, the secondary structure of the peptides in the liposome lipid membranes was probed by circular dichroism. The results showed that CNY21 binds to zwitterionic and anionic lipid membranes, and that CNY21 shows a higher binding tendency than CNY21 R-S. Furthermore, CNY21 variants results in pore formation and leakage of the liposomes, with an efficiency in the order CNY21H-L≈CNY21H-K>CNY21>CNY21H-P≧CNY21 R-S. Also CD indicated presence of helix structure of peptides in the liposome lipid membrane to an extent decreasing in the same order.

### Example 10

### Peptides

Peptides were from Sigma-Genosys, generated by a peptide synthesis platform (PEPscreen®, Custom Peptide Libraries, SigmaGenosys). Yield was ˜1-6 mg, and peptide length 20 amino acids. MALDI-ToF Mass Spectrometry was performed on these peptides. Average Crude Purity of 20mers was ˜61%. Peptides were supplied lyophilized and in a 96-well tube rack. Prior to biological testing the PEP-screen peptides were diluted in dH₂0 (5 mM stock), and stored at −20 C. This stock solution was used for the subsequent experiments.

Microorganisms.

Escherichia coli 37.4 isolate was originally obtained from a patient with a chronic venous ulcer, while Staphylococcus aureus isolate BD14312 was from a patient with atopic dermatitis. Staphylococcus aureus ATCC29213 and Candida albicans ATCC90028 were both obtained from the Clinical Bacteriology Department at Lund University hospital.

Radial Diffusion Assay.

Essentially as described earlier (Lehrer et al., J Immunol Methods 137, 167-173, 1991, Andersson et al., Eur J Biochem 271, 1219-1226, 2004) bacteria were grown to mid-logarithmic phase in 10 ml of full-strength (3% w/v) trypticase soy broth (TSB) (Becton-Dickinson, Cockeysville, Md.). The microorganisms were then washed once with 10 mM Tris, pH 7.4.4×10⁶ bacterial colony forming units were added to 15 ml of the underlay agarose gel, consisting of 0.03% (w/v) TSB, 1% (w/v) low electroendosmosis type (EEO) agarose (Sigma, St Louis Mo.) and 0.02% (v/v) Tween 20 (Sigma). The underlay was poured into a ø 144 mm petri dish. After agarose solidification, 4 mm-diameter wells were punched and 6 μl of test sample was added to each well. Plates were incubated at 37° C. for 3 hours to allow diffusion of the peptides. The underlay gel was then covered with 15 ml of molten overlay (6% TSB and 1% Low-EEO agarose in dH₂O). Antimicrobial activity of a peptide is visualized as a zone of clearing around each well after 18-24 hours of incubation at 37° C.

### Example 11

### Hemolysis Assay

EDTA-blood was centrifuged at 800 g for 10 min, whereafter plasma and buffy coat were removed. The erythrocytes were washed three times and resuspended in 5% PBS, pH 7.4. The cells were then incubated with end-over-end rotation for 1 h at 37° C. in the presence of peptides (60 mM). 2% Triton X-100 (Sigma-Aldrich) served as positive control. The samples were then centrifuged at 800 g for 10 min. The absorbance of hemoglobin release was measured at λ 540 nm and is in the plot expressed as % of TritonX-100 induced hemolysis.

### Example 12

### Prediction of Helix Formation

An algorithm based on helix-coil transition theory, AGADIR, was used to predict helical propensity (Lacroix et al., J Mol Biol 284,173-191, 1998). Calculation was performed by submitting peptide sequences to the EMBL WWW gateway to AGADIR service (littp://www.emblheidelberg.de/Services/serrano/agadir/agadir-start.html). Input parameters were as follow: Cterm free, Nterm free, pH 7.4, Temperature 278 K and Ionic Strength 0.15 M.

### Results Obtained from Example 10-12

Based on the criteria defined in EXAMPLE 1 several new variants with increased positive net charge, high helicity and increased amphipathicity were designed and synthesized. In order to rationally test as many different variants as possible a PEP screen library was utilized as described above. The C-terminal arginine residue in CNY21 was omitted because the PEPscreen library only complies with 20-mer peptides. The CNY20 variant “CNYITELRRQHARASHLGLA” was compared with CNY21 in RDA assays using the two clinical isolates Pseudomonas aeruginosa 15159 and Escherichia coli 37.4. Following data were obtained for P. aeruginosa using 100 μM of peptides (n=9); mean value; CNY21: 5.00, SD; 0.81, SEM 0.27. CNY 20: mean value; 6.02, SD; 0.59, SEM; 0.20. E. coli; CNY21: mean value 6.02, SD 0.93, SEM; 0.31, CNY20: mean value; 8.03, SD; 1.22, SEM, 0.41. Hence, no decrease in activity was noted for CNY20 when compared with CNY21. Peptides number 2 to 17 were designed to increase helicity by varying the N-cap, N-cap+3 and N-cap+4 positions (Table 1, EXAMPLE 1). Peptides number 18-20 were designed to stabilize helicity by varying position C-cap−4 and C-cap−2 (Table 1, EXAMPLE 1). For the peptides ranging from 21 to 30 the spacing between leucines was varied in order to increase helicity (Table 1, EXAMPLE 1). The amphipathic structure was further optimized by replacing amino acids at positions 8, 11, 15, 16 and 17 in peptides 31 to 43 (Table 1, EXAMPLE 1). For peptides 44 to 56, optimal amphipathicity and increased helicity was obtained by combining the previously described substitutions. Finally, peptides 57 to 74 were designed to increase the net positive charge in combination with stabilized helicity and optimal amphipathicity.

The results from the biological testing are displayed in Table 1. Among the first 20 peptides, which were designed to increase helicity by stabilization of N-cap and C-cap motifs, relatively few displayed any enhancement of antimicrobial effects. Only the peptides with net charge+3 displayed significantly increased RDA values (i.e., peptide no. 14). None of these peptides were hemolytic. The more hydrophobic peptides no. 21-30 displayed similar effects. Peptides no. 27-30 were not included in the study. The peptides with optimized amphipathic structure demonstrated high RDA values (peptides 39, 40, 42 and 43). However, peptides no. 42 and 43, predicted to have a high helical content, also displayed a high hemolytic activity. Most of peptides 44-56 which had combined substitutions to maximize helicity demonstrated a high hemolytic activity. Peptides no. 51 and 53 were omitted from the study due to solubility problems. Peptide no. 47 displayed a high antimicrobial activity paired with a low hemolytic activity. Peptides 57-74, except for peptide no. 73, were hemolytic, possibly due to their high net charge and helicity. Notably, however, peptide no. 73, having the highest net charge in the series, displayed a significant antimicrobial effect against S. aureus and Candida and exerted a low hemolytic activity.

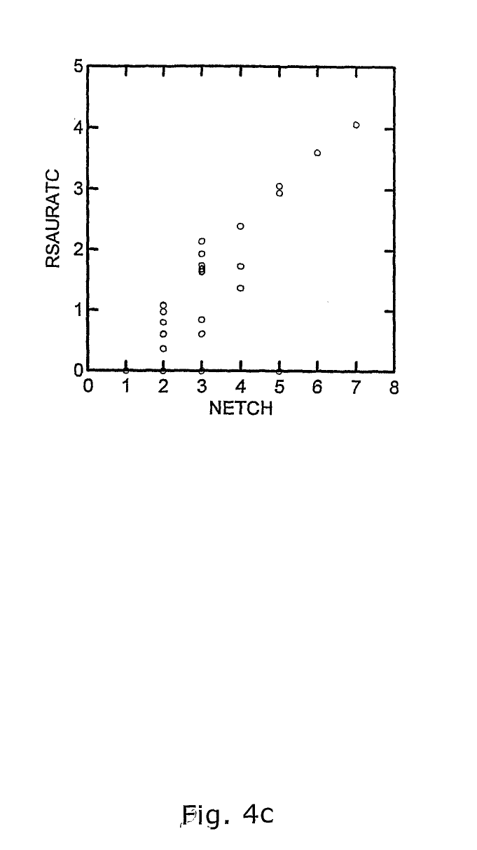

No clear correlation to net charge or helicity was observed for the E. coli RDA values. A low correlation to net charge and high predicted helicity was detected for the hemolytic activity. On the other hand, the RDA values for S. aureus and Candida correlated strongly to net charge. Peptides with high positive net charge displayed a high antimicrobial activity (FIG. 4).

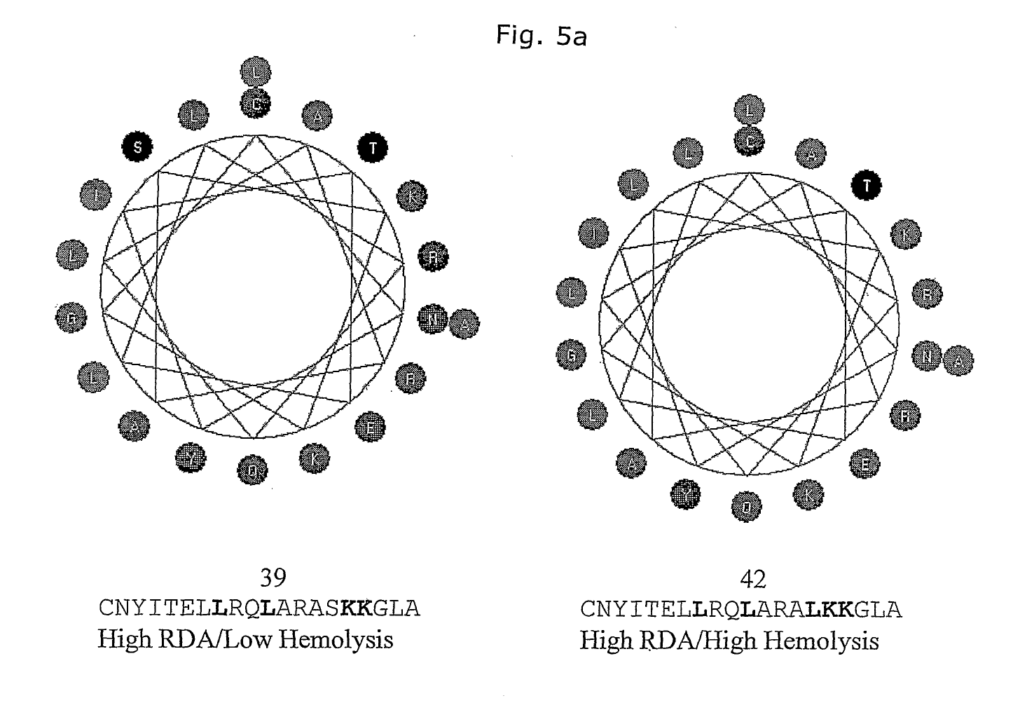

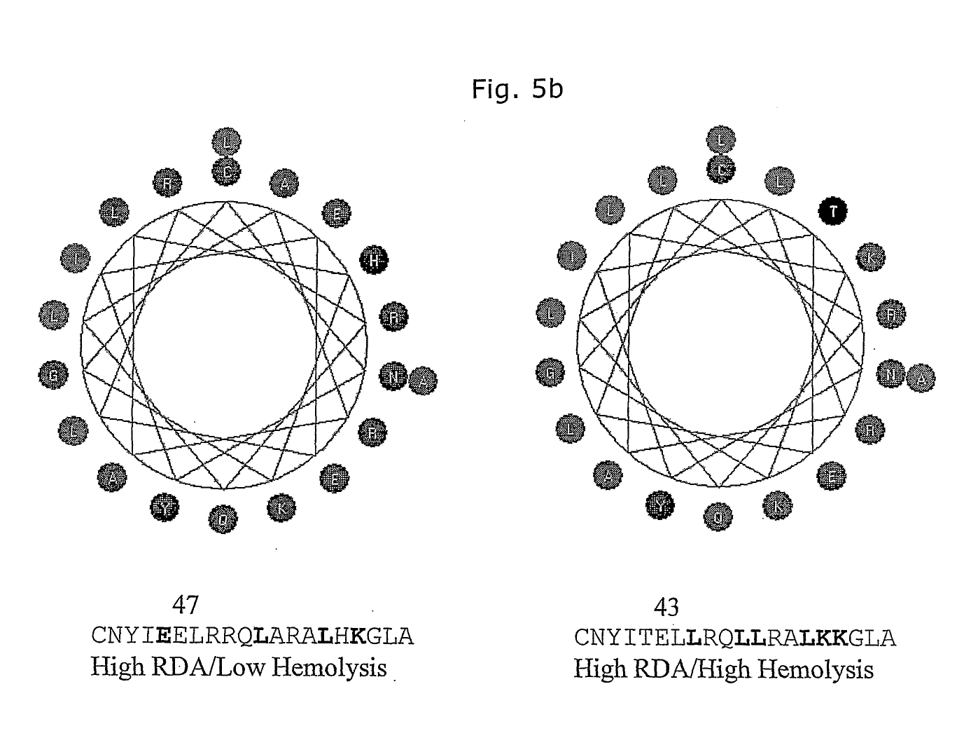

Notably, peptides with high predicted helicity and high antimicrobial activity displayed significant differences in hemolytic activity. For example, peptides no. 39 and 47 displayed low hemolytic activity whereas peptides no. 42 and 43 were strongly hemolytic. Remarkably, peptide 39 and 42 only differ by one amino acid, where peptide 42 has an additional substitution of serine to leucine at position 15. Possibly, the large difference in hemolytic activity reflects the fact that peptide 42 forms a more optimal amphipathic helix (FIG. 5). The same reasoning applies to peptides 43 and 47. Peptide 43 has arginine 8 substituted by leucine (FIG. 5).

Second Generation CNY20 Peptides and their Activities

Four of the variants in the first PEPscreen demonstrating high antimicrobial activities against E. coli in RDA paired with a low hemolytic activity comprised imperfect amphipathic helices. Therefore, additional variants were designed with amino acid substitutions yielding a break in the structural organization of the amphipathic peptides. The net charge was maintained around +2 to +3, and peptides were designed to have a relatively high (but not exceedingly high) helical content (20-60%).

New variants were designed with a break of amphipathicity in the N-terminal region (140-146), in the C-terminal region (147-160), or in the central region (161-168). Additional peptides had a high positive net charge (169-171), a high hydrophobicity (172-177), contained acetylated and amidated N- and C-terminus (179-181), or comprised all D-amino acids (182-184) (Table 2).

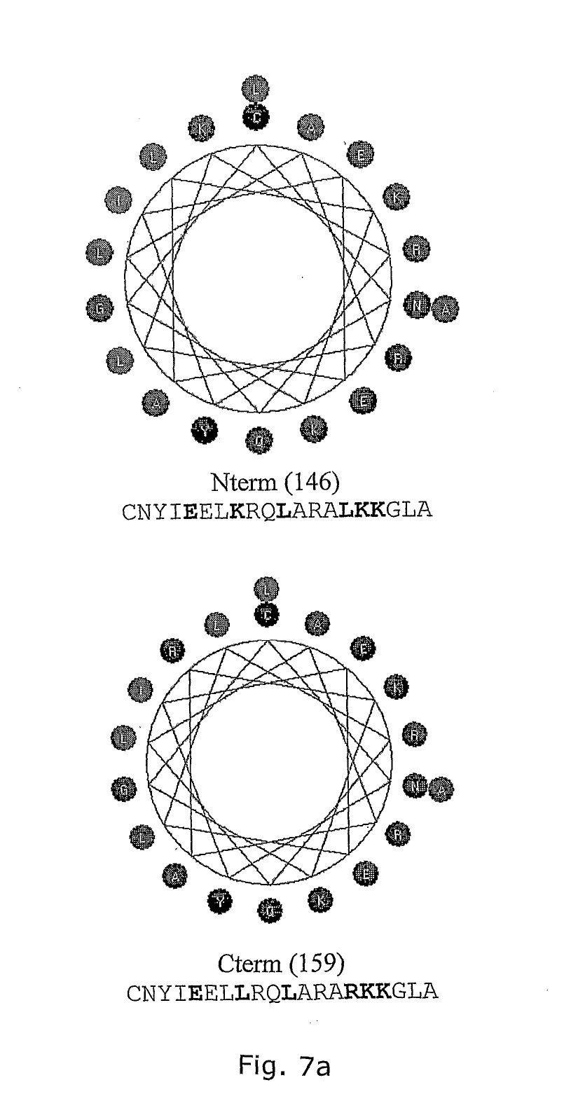

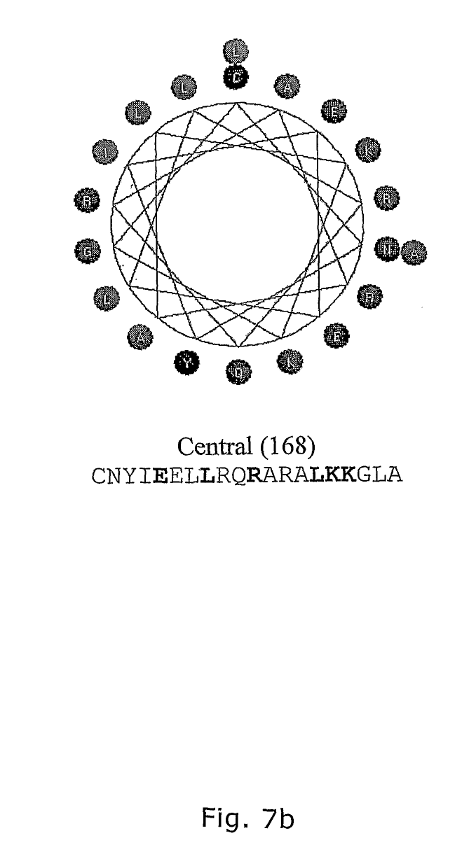

In order to enhance amphipathicity in the C-terminus of peptides, substitutions of polar amino acids with leucines were performed at positions 11 and 15 (FIGS. 6 and 4). Furthermore, substitutions of hydrophobic amino acids to lysines were made at positions 16 and 17 (FIGS. 6 and 7). To enhance amphipathicity in the N-terminal region substitutions to leucine were made at positions 8 and 11 (FIGS. 6 and 7). Peptides with a break of amphipathicity in the central region all had a positively charged arginine or lysine at position 11, hydrophobic leucine at positions 8 and 15 and a lysine at position 17. All these variants were also combined with a threonine to glutamate substitution in position 5 in order to increase helicity by stabilization of the capping motif as described in EXAMPLE 1.

Almost all peptides tested displayed significant antimicrobial effects against E. coli and S. aureus. No obvious differences were detected for the different peptide groups having breaks in amphipathicity at the N-terminus, C-terminus or central regions. However, the results suggested that peptides with breaks in the central region were slightly more antimicrobial against S. aureus. Peptides with a higher net charge also displayed a better antimicrobial effect. Acetylation and amidation in the N- and C-terminus had little effect and the D-amino acid peptides showed similar antimicrobial effects as peptides composed of L-amino acids. Peptides with glutamate in position 5 were not more antimicrobial than peptides not substituted in this position. Likely, the stabilization of helicity was out-weighted by the decrease in net charge.

Peptides with good antimicrobial activity contained threonine or glutamate at position 5, arginine, lysine or leucine at position 8, leucine, arginine or lysine at position 11, alanine or leucine at position 12, alanine or leucine at position 14, serine, leucine, arginine or lysine at position 15, histidine or lysine at position 16, and leucine or lysine at position 17. Particularly, peptides no. 140, 146 and 160 showed high antimicrobial activity against E. coli, peptides no. 160, 161 and 165 showed high antimicrobial activity against S. aureus and peptides no. 158, 160 and 171 displayed high antimicrobial activity against both E. coli and S. aureus. Furthermore, the results indicate that peptides with imperfect amphipathic structures, and a high net charge are preferably active on both S. aureus as well as Candida species.

Concludingly, the study shows that a low number of amino acid substitutions at well-defined positions of the CNY20 peptide can increase the antimicrobial activity but preserve a low hemolytic activity, thus yielding peptides with a higher therapeutic index than the original peptide. A combination of a relatively high net charge, a propensity to adopt an α-helical conformation and a not too perfect amphipathicity are important factors for reaching this. Substitutions at positions 8, 11, and 17 were found to be especially essential for the CNY20 peptide.

TABLE 1

Activities of CNY variants against E. coli 37.4, S. aureus BD14312, S. aureus
ATCC29213, Candida ATCC90028 and measurement of hemolytic activity. The anti-
microbial effects correspond to zones of inhibition (in mm) and hemolysis is
expressed as % of total hemolysis. The Agadir value is calculated as described
in EXAMPLE 1.

RDA (mm)

| | | |
|---|---|---|
| E. coli | S. aureus | Candida |

| | | | | | |
|---|---|---|---|---|---|
| Net | 37.4 | BD14312 | ATCC29213 | ATCC90028 | % |

| | | | | | | | | | | | | |
|---|---|---|---|---|---|---|---|---|---|---|---|---|
| No. | Sequence | Agadir | charge | Mean | SD | Mean | SD | Mean | SD | Mean | SD | Hemolysis |

| | | | | | | | | | | | | |
|---|---|---|---|---|---|---|---|---|---|---|---|---|
| 1. | CNYITELRRQHARASHLGLA | 4.99 | +2 | 1.750 | 0.200 | 0 | 0.000 | 0 | 0.000 | 0.00 | 0.00 | 2.752 |
| 2. | CNYIDELRRQHARASHLGLA | 12.04 | +1 | 0.300 | 0.173 | 0 | 0.000 | 0 | 0.000 | 0.00 | 0.00 | 2.143 |
| 3. | CNYIEELRRQHARASHLGLA | 15.21 | +1 | 0.000 | 0.000 | 0 | 0.000 | 0 | 0.000 | 0.00 | 0.00 | 2.262 |
| 4. | CNYIKELRRQHARASHLGLA | 1.80 | +3 | 2.667 | 0.404 | 0.91 | 0.155 | 0.605 | 0.105 | 0.00 | 0.00 | 3.312 |
| 5. | CNYIRELRRQHARASHLGLA | 2.14 | +3 | 2.967 | 0.685 | 1.145 | 0.135 | 0.84 | 0.135 | 2.61 | 0.33 | 2.859 |
| 6. | CNYIEELRRQHARASHLGLA | 15.21 | +1 | 0.000 | 0.000 | 0 | 0.000 | 0 | 0.000 | 0.00 | 0.00 | 1.935 |
| 7. | CNYIDELRRQHARASHLGLA | 12.04 | +1 | 0.000 | 0.000 | 0 | 0.000 | 0 | 0.000 | 0.00 | 0.00 | 1.979 |
| 8. | CSYITELRRQHARASHLGLA | 5.10 | +2 | 2.300 | 0.557 | 0 | 0.000 | 0 | 0.000 | 0.00 | 0.00 | 2.671 |
| 9. | CSYIEELRRQHARASHLGLA | 19.61 | +1 | 0.000 | 0.000 | 0 | 0.000 | 0 | 0.000 | 0.00 | 0.00 | 1.917 |
| 10. | CSYIDELRRQHARASHLGLA | 14.40 | +1 | 0.000 | 0.000 | 0 | 0.000 | 0 | 0.000 | 0.00 | 0.00 | 2.224 |
| 11. | CTYITELRRQHARASHLGLA | 3.28 | +2 | 0.750 | 0.503 | 0 | 0.000 | 0 | 0.000 | 0.00 | 0.00 | 2.884 |
| 12. | CTYIEELRRQHARASHLGLA | 15.35 | +1 | 0.000 | 0.000 | 0 | 0.000 | 0 | 0.000 | 0.00 | 0.00 | 2.212 |
| 13. | CTYIDELRRQHARASHLGLA | 11.97 | +1 | 0.100 | 0.152 | 0 | 0.000 | 0 | 0.000 | 0.00 | 0.00 | 2.011 |
| 14. | CNYITALRRQHARASHLGLA | 4.68 | +3 | 3.483 | 0.516 | 1.25 | 0.189 | 0.97 | 0.189 | 3.53 | 1.32 | 2.878 |
| 15. | CNYIEELRRQHARASHLGLA | 15.21 | +1 | 0.000 | 0.000 | 0 | 0.000 | 0 | 0.000 | 0.00 | 0.00 | 1.653 |
| 16. | CNYIEALRRQHARASHLGLA | 16.19 | +2 | 1.950 | 0.305 | 0 | 0.000 | 0 | 0.000 | 0.00 | 0.00 | 2.583 |
| 17. | CNYIELLRRQHARASHLGLA | 17.28 | +2 | 2.250 | 0.300 | 0 | 0.000 | 0 | 0.000 | 0.00 | 0.00 | 2.369 |
| 18. | CNYITELRRQHARLSHLGLA | 5.14 | +2 | 2.833 | 0.404 | 0 | 0.00 | 0 | 0.000 | 0.15 | 0.17 | 2.457 |
| 19. | CNYITELRRQHARASALGLA | 5.90 | +2 | 0.000 | 0.000 | 0 | 0.00 | 0 | 0.000 | 0.00 | 0.00 | 2.727 |
| 20. | CNYITELRRQHARLSALGLA | 6.47 | +2 | 0.250 | 0.360 | 0.155 | 0.00 | 0 | 0.000 | 0.00 | 0.00 | 2.451 |
| 21. | CNYITELLRQHARASHLGLA | 10.98 | +1 | 2.367 | 0.521 | 0.065 | 0.00 | 0 | 0.000 | 0.00 | 0.00 | 1.301 |
| 22. | CNYITELRRQLARASHLGLA | 15.65 | +2 | 2.550 | 0.573 | 0.24 | 0.00 | 0 | 0.000 | 0.68 | 0.10 | 2.212 |
| 23. | CNYITELRRQHLRASHLGLA | 5.27 | +2 | 1.583 | 0.578 | 0 | 0.00 | 0 | 0.000 | 0.00 | 0.00 | 2.664 |
| 24. | CNYITELRRQHARASLLGLA | 5.91 | +2 | 2.483 | 0.578 | 0 | 0.00 | 0 | 0.000 | 0.00 | 0.00 | 2.664 |
| 25. | CNYITELLRQLARASHLGLA | 32.88 | +1 | 2.267 | 0.682 | 0 | 0.000 | 0 | 0.000 | 0.00 | 0.00 | 1.024 |
| 26. | CNYITELLRQLLRASHLGLA | 41.17 | +1 | 2.440 | 0.431 | 0 | 0.000 | 0 | 0.000 | 0.00 | 0.00 | 1.533 |
| 27. | CNYITELRRQLARASLLGLA* | 8.05 | +2 | 0.000 | 0.00 | 0 | 0.000 | 0 | 0.000 | 0.00 | 0.00 | 0.000 |
| 28. | CNYITELRRQLLRASLLGLA* | 22.18 | +2 | 0.000 | 0.00 | 0 | 0.000 | 0 | 0.000 | 0.00 | 0.00 | 0.000 |
| 29. | CNYITELLRQLARASLLGLA* | 36.42 | +1 | 0.000 | 0.00 | 0 | 0.000 | 0 | 0.000 | 0.00 | 0.00 | 0.000 |
| 30. | CNYITELLRQLLRASLLGLA* | 48.42 | +1 | 0.000 | 0.00 | 0 | 0.000 | 0 | 0.000 | 0.00 | 0.00 | 0.000 |
| 31. | CNYITELLRQHARASHLGLA | 10.98 | +1 | 2.450 | 0.569 | 0 | 0.000 | 0 | 0.000 | 0.00 | 0.00 | 1.332 |
| 32. | CNYITELLRQLARASHLGLA | 32.88 | +1 | 2.667 | 0.425 | 0 | 0.000 | 0 | 0.000 | 0.00 | 0.00 | 1.056 |
| 33. | CNYITELLRQLARALHLGLA | 47.43 | +1 | 0.000 | 0.00 | 0 | 0.000 | 0 | 0.000 | 0.00 | 0.00 | 3.418 |
| 34. | CNYITELRRQHARASHKGLA | 5.25 | +3 | 1.383 | 0.704 | 0 | 0.000 | 0 | 0.000 | 1.82 | 0.57 | 1.931 |
| 35. | CNYITELLRQLARALHKGLA | 49.61 | +2 | 2.800 | 0.300 | 0.09 | 0.072 | 0.795 | 0.540 | 0.00 | 0.00 | 7.793 |
| 36. | CNYITELRRQHARASKLGLA | 6.12 | +3 | 2.350 | 0.306 | 0 | 0.000 | 0 | 0.000 | 0.00 | 0.00 | 2.797 |
| 37. | CNYITELLRQHARASKLGLA | 12.99 | +2 | 1.550 | 0.703 | 0 | 0.000 | 0 | 0.000 | 0.00 | 0.00 | 1.342 |
| 38. | CNYITELLRQLARASKLGLA | 37.40 | +2 | 2.400 | 0.514 | 0 | 0.000 | 0 | 0.000 | 1.12 | 0.34 | 1.663 |
| 39. | CNYITELLRQLARASKKGLA | 38.91 | +3 | 4.250 | 2.004 | 0.495 | 0.051 | 0 | 0.000 | 4.36 | 0.46 | 3.639 |
| 40. | CNYITELLRQLARASQKGLA | 37.80 | +2 | 3.283 | 0.834 | 0 | 0.000 | 0 | 0.000 | 0.00 | 0.00 | 1.349 |
| 41. | CNYITELLRQLARASEKGLA | 33.86 | +1 | 0.000 | 0.000 | 0 | 0.000 | 0 | 0.000 | 0.00 | 0.00 | 1.131 |
| 42. | CNYITELLRQLARALKKGLA | 58.58 | +3 | 4.117 | 0.755 | 0.925 | 0.135 | 1.65 | 0.135 | 3.75 | 0.60 | 10.078 |
| 43. | CNYITELLRQLLRALKKGLA | 66.88 | +3 | 4.467 | 0.517 | 1.105 | 0.225 | 1.63 | 0.225 | 3.37 | 0.11 | 20.199 |
| 44. | CNYIEELLRQLARALHKGLA | 60.35 | +1 | 2.183 | 1.173 | 0 | 0.000 | 0 | 0.000 | 2.07 | 0.32 | 1.785 |
| 45. | CSYIEELLRQLARALHKGLA | 63.75 | +1 | 0.000 | 0.00 | 0 | 0.000 | 0 | 0.000 | 0.00 | 0.00 | 2.658 |
| 46. | CSYIEELLRQLLRALLKGLA | 79.13 | +1 | 0.000 | 0.00 | 0 | 0.000 | 0 | 0.000 | 0.00 | 0.00 | 8.145 |
| 47. | CNYIEELRRQLARALHKGLA | 52.46 | +2 | 4.883 | 1.200 | 0 | 0.228 | 0 | 0.228 | 3.29 | 1.53 | 2.313 |
| 48. | CNYIEELRRQLLRALHKGLA | 59.49 | +2 | 2.833 | 0.506 | 0 | 0.256 | 0 | 0.256 | 3.07 | 0.08 | 2.834 |
| 49. | CSYIEELRRQLARALHKGLA | 57.05 | +2 | 3.850 | 0.710 | 0 | 0.207 | 1.075 | 0.207 | 0.00 | 0.00 | 5.474 |
| 50. | CSYIEELRRQLLRALLKGLA | 75.90 | +2 | 0.000 | 0.00 | 0 | 0.000 | 0.36 | 0.000 | 0.00 | 0.00 | 2.100 |
| 51. | CSYIEELRRQLLRALLRGLA* | 76.36 | +2 | 0.000 | 0.00 | 0 | 0.000 | 0 | 0.000 | 0.00 | 0.00 | 0.000 |
| 52. | CNYIEELRRQLLRALLKGLA | 73.62 | +2 | 0.000 | 0.00 | 0 | 0.425 | 0 | 0.000 | 0.00 | 0.00 | 2.484 |
| 53. | CNYIEELRRQLLRALLRGLA* | 74.07 | +2 | 0.000 | 0.00 | 0 | 0.000 | 0 | 0.000 | 0.00 | 0.00 | 0.000 |
| 54. | CNYIEELLRQLLRALKKGLA | 72.89 | +2 | 2.433 | 0.710 | 0.54 | 0.036 | 0 | 0.000 | 0.00 | 0.00 | 4.470 |
| 55. | CSYIEELLRQLLRALKKGLA | 74.48 | +2 | 0.000 | 0.000 | 0.7 | 0.150 | 1.08 | 0.150 | 1.86 | 0.12 | 7.849 |
| 56. | CSYIEELLRQLLRALKRGLA | 74.68 | +2 | 1.700 | 0.361 | 0.07 | 0.179 | 0.595 | 0.179 | 1.63 | 0.34 | 11.155 |
| 57. | CKYITELLRQLARALKLGLA | 41.93 | +3 | 2.817 | 0.604 | 0.675 | 0.512 | 1.93 | 0.512 | 3.24 | 0.52 | 13.876 |
| 58. | CNYITELLRQLARALKLGLA | 57.45 | +2 | 2.650 | 0.461 | 0 | 0.000 | 0.605 | 0.032 | 1.82 | 0.34 | 7.686 |
| 59. | CNYITELRRQLARALKLGLA | 37.65 | +3 | 0.000 | 0.000 | 1.01 | 0.369 | 1.675 | 0.369 | 5.58 | 0.51 | 2.903 |
| 60. | CNYITELLRQLARALKRGLA | 58.57 | +3 | 1.567 | 0.787 | 0.72 | 0.279 | 1.69 | 0.279 | 3.52 | 0.27 | 11.124 |
| 61. | CSYIELLRRQLARALHLGLA | 56.06 | +2 | 2.467 | 0.452 | 0.55 | 0.684 | 0.605 | 0.071 | 1.96 | 0.15 | 5.099 |
| 62. | CSYIELLRRQLARALHRGLA | 57.69 | +3 | 2.833 | 0.326 | 1.16 | 0.745 | 2.14. | 0.659 | 5.03 | 0.62 | 8.019 |
| 63. | CSYIELLRRQLARALLRGLA* | 67.43 | +3 | 0.000 | 0.000 | 0 | 0.000 | 0 | 0.000 | 0.00 | 0.00 | 0.000 |
| 64. | CSYIELLRRQLARALKRGLA | 65.58 | +4 | 3.300 | 0.957 | 1.49 | 0.325 | 2.39 | 0.325 | 4.57 | 1.12 | 12.060 |
| 65. | CSYIELLRRQLLRALKRGLA | 70.34 | +4 | 2.417 | 0.616 | 1.17 | 0.108 | 1.365 | 0.108 | 4.90 | 1.32 | 12.016 |
| 66. | CKYIELLRRQLARALKRGLA | 61.04 | +5 | 0.833 | 0.517 | 2.725 | 0.724 | 3.055 | 0.724 | 6.31 | 0.22 | 12.267 |
| 67. | CKYIELLRRQLLRALKRGLA | 67.77 | +5 | 2.983 | 0.616 | 0 | 0.000 | 0 | 0.000 | 0.00 | 0.00 | 19.275 |
| 68. | CKYIELLRRQLLRALKRGLR | 68.59 | +6 | 3.067 | 0.569 | 2.93 | 0.298 | 3.605 | 0.298 | 5.48 | 0.25 | 23.976 |
| 69. | CSYIELLRRQLLRALKRGLR | 71.03 | +5 | 2.850 | 0.460 | 2.235 | 0.260 | 2.94 | 0.260 | 4.79 | 0.24 | 19.765 |
| 70. | CSYIEELRRQLLRALKRGLR | 70.36 | +4 | 1.850 | 0.462 | 0.99 | 0.211 | 1.73 | 0.211 | 5.94 | 0.24 | 12.525 |
| 71. | CSYIEELLRQLLRALKRGLR | 75.24 | +3 | 0.450 | 0.626 | 0.82 | 0.384 | 1.74 | 0.384 | 2.71 | 0.37 | 12.619 |
| 72. | CSYIEELLRQLLRALLRGLR | 80.29 | +2 | 0.000 | 0.000 | 0 | 0.000 | 0 | 0.000 | 0.00 | 0.00 | 20.538 |
| 73. | CKYITLLRRQLLRALKRGLR | 33.11 | +7 | 2.450 | 0.612 | 5.79 | 0.386 | 4.065 | 0.386 | 5.38 | 0.93 | 2.382 |
| 74. | CNYITELRRQHARASHLGLR | 5.05 | +3 | 0.567 | 0.460 | 0.37 | 0.290 | 0 | 0.000 | 3.32 | 0.22 | 2.991 |
| 75. | CNYITELRRQHARASHLGLA | 4.99 | +2 | 1.767 | 0.557 | 0 | 0.000 | 0 | 0.000 | 0.77 | 0.25 | 2.294 |
| 76. | LL37 | | | 3.529 | 0.719 | 1.285 | | 2.215 | 0.582 | 2.43 | 1.35 | 18.933 |
| 77. | pos. control in | | | | | | | | | | | 100.00 |
| | hemolysis | | | | | | | | | | | |

All values are the mean value from three different measurements except for the E. coli data, which are mean values from six measurements.
Peptides labeled with * were not included in the study due to solubility problems.

TABLE 2

Second generation PEPscreen CNY20 variants. RDA analysis was
performed using E. coli 37.4 and S. aureus ATCC29213. The
antimicrobial effects correspond to zones of inhibition (in mm)
and hemolysis is expressed as % of total hemolysis. The Agadir
value is calculated as described in EXAMPLE 1.

RDA (mm)

| | |
|---|---|
| E. coli | S. aureus |

| | | | Net | 37.4 | | ATCC29213 | % Hemolysis | |
|---|---|---|---|---|---|---|---|---|
| No. | Sequence | Agadir | Charge | Mean | SD | Mean | Mean | SD |

| | | | | | | | | |
|---|---|---|---|---|---|---|---|---|
| 139 | CNYITELRRQHARASHLGLA | 4.99 | +2 | 3.960 | 0.325 | 1.21 | 2.745 | 0.004 |
| 140 | CNYITELRRQLARALHKGLA | 29.31 | +3 | 6.460 | 1.032 | 3.81 | 5.145 | 0.004 |
| 141 | CNYITELRRQLARALKKGLA | 39.65 | +4 | 5.350 | 0.849 | 3.27 | 17.716 | 0.904 |
| 142 | CNYIEELRRQLARALKKGLA | 61.26 | +3 | 5.685 | 0.827 | 2.71 | 3.734 | 0.009 |
| 143 | CNYITELKRQLARALHKGLA | 27.12 | +3 | 5.865 | 1.308 | 3.15 | 3.450 | 0.019 |
| 144 | CNYITELKRQLARALKKGLA | 37.41 | +4 | 5.570 | 0.651 | 3.85 | 3.597 | 0.023 |
| 145 | CNYIEELKRQLARALHKGLA | 49.03 | +2 | 5.695 | 0.643 | 2.36 | 2.495 | 0.003 |
| 146 | CNYIEELKRQLARALKKGLA | 58.62 | +3 | 6.140 | 0.184 | 4.35 | 2.521 | 0.009 |
| 147 | CNYITELLRQLARASHKGLA | 34.04 | +2 | 5.070 | 0.198 | 4.98 | 2.555 | 0.017 |
| 148 | CNYIEELLRQLAPASHKGLA | 45.86 | +1 | 2.900 | 0.184 | 0.99 | 1.454 | 0.002 |
| 149 | CNYIEELLRQLARASKKGLA | 50.63 | +2 | 4.260 | 0.339 | 1.1 | 1.506 | 0.008 |
| 150 | CNYITELLRQLARATHKGLA | 29.80 | +2 | 5.200 | 0.707 | 1.98 | 3.777 | 0.017 |
| 151 | CNYIEELLRQLARATHKGLA | 41.74 | +1 | 4.845 | 0.120 | 0.98 | 1.626 | 0.015 |
| 152 | CNYITELLRQLARATKKGLA | 34.83 | +3 | 5.280 | 1.160 | 4.85 | 3.984 | 0.040 |
| 153 | CNYIEELLRQLARATKKGLA | 47.15 | +2 | 4.655 | 0.926 | 2.88 | 1.755 | 0.014 |
| 154 | CNYITELLRQLARANKKGLA | 40.93 | +3 | 5.105 | 1.223 | 0.98 | 2.564 | 0.019 |
| 155 | CNYITELLRQLARAQKKGLA | 47.11 | +3 | 4.840 | 0.679 | 3.5 | 5.817 | 0.128 |
| 156 | CNYITELLRQLARARHKGLA | 44.93 | +3 | 5.140 | 0.566 | 5.71 | 3.321 | 0.020 |
| 157 | CNYIEELLRQLARARHKGLA | 56.18 | +2 | 3.550 | 0.863 | 4.95 | 1.282 | 0.011 |
| 158 | CNYITELLRQLARARKKGLA | 52.32 | +4 | 5.860 | 0.255 | 5.73 | 3.829 | 0.013 |
| 159 | CNYIEELLRQLARARKKGLA | 62.48 | +3 | 5.165 | 0.431 | 3.25 | 1.841 | 0.004 |
| 160 | CNYITELLRQLARAKKKGLA | 50.90 | +4 | 9.630 | 0.127 | 7.2 | 2.512 | 0.009 |
| 161 | CNYITELLRQKARALHKGLA | 23.53 | +3 | 4.703 | 0.805 | 8.61 | 1.902 | 0.008 |
| 162 | CNYIEELLRQKARALHKGLA | 36.29 | +2 | 5.587 | 0.064 | 2.36 | 2.056 | 0.011 |
| 163 | CNYITELLRQKARALKKGLA | 30.90 | +4 | 0.000 | 0.000 | 5.23 | 2.478 | 0.011 |
| 164 | CNYIEELLRQKARALKKGLA | 44.94 | +3 | 3.940 | 0.485 | 5.59 | 2.340 | 0.012 |
| 165 | CNYITELLRQRARALHKGLA | 25.83 | +3 | 3.977 | 0.345 | 7.03 | 2.659 | 0.017 |
| 166 | CNYIEELLRQRARALHKGLA | 38.84 | +2 | 3.513 | 0.133 | 0 | 2.117 | 0.017 |
| 167 | CNYITELLRQRARALKKGLA | 34.42 | +4 | 4.487 | 0.427 | 3.98 | 2.642 | 0.009 |
| 168 | CNYIEELLRQRARALKKGLA | 48.35 | +3 | 4.460 | 0.298 | 4.15 | 2.573 | 0.054 |
| 169 | CNYITELRRQKLRLLKKGLA | 26.19 | +5 | 4.370 | 0.767 | 5.63 | 2.512 | 0.013 |
| 170 | CNYITELRRQKLRLLHKGLA | 17.96 | +4 | 4.650 | 0.575 | 1.49 | 2.211 | 0.011 |
| 171 | CNYIKELLRQKLRASHLGLA | 10.66 | +3 | 5.660 | 0.691 | 5.38 | 2.383 | 0.008 |
| 172 | CNYIEELRRQLARALHKGLA | 52.46 | +2 | 5.230 | 0.406 | 2.84 | 2.323 | 0.001 |
| 173 | CNYIEELRRQLARALHKWLA | 52.26 | +2 | 4.057 | 0.189 | 6.03 | 8.148 | 0.031 |
| 174 | CNYIEELRRQLARAWHKGLA | 42.88 | +2 | 3.970 | 0.631 | 2.68 | 2.056 | 0.004 |
| 175 | CNYIEELRRQWARALHKGLA | 32.28 | +2 | 3.680 | 0.525 | 0 | 1.824 | 0.006 |
| 176 | CNYIEELWRQLARALHKGLA | 26.64 | +1 | 2.000 | 0.056 | 0.79 | 2.771 | 0.016 |
| 177 | CNYIWELRRQLARALHKGLA | 23.17 | +3 | 4.710 | 0.328 | 0 | 3.175 | 0.011 |
| 178 | CNYITELRRQHARASHLGLA | 4.99 | +2 | 2.977 | 0.470 | 4.52 | 1.988 | 0.012 |
| 179 | acetyl- | | | 2.677 | 0.284 | 4.03 | 2.495 | 0.026 |
| | CNYITELRRQHARASHLGLA- | | | | | | | |
| | amide | | | | | | | |
| 180 | acetyl- | | | 5.933 | 1.291 | 0 | 5.447 | 0.023 |
| | CNYITELLRQLARASKKGLA- | | | | | | | |
| | amide | | | | | | | |
| 181 | acetyl- | | | 4.330 | 1.546 | 4.24 | 4.861 | 0.036 |
| | CNYIEELRRQLARALHKGLA- | | | | | | | |
| | amide | | | | | | | |
| 182 | CNYITELRRQHARASHLGLA | | | 4.503 | 0.394 | 4.37 | 2.659 | 0.021 |
| | (all d-amino acids) | | | | | | | |
| 183 | CNYITELLRQLARASKKGLA | | | 4.617 | 0.953 | 5.83 | 5.128 | 0.021 |
| | (all d-amino acids) | | | | | | | |
| 184 | CNYIEELRRQLARALHKGLA | | | 2.957 | 0.186 | 3.23 | 3.235 | 0.007 |
| | (all d-amino acids) | | | | | | | |
| LL37 | | | | 5.604 | 0.610 | 8.755 | 16.013 | 0.060 |
| | Control (+) | | | | | | 100.000 | 0.010 |
| | Control (−) | | | | | | 2.366 | 0.020 |

1. Use of a peptide comprising the SEQ ID: 1 or analogue thereof, wherein said peptide differs from the amino acid sequence shown in SEQ ID NO:1 in that at least one amino acid residue selected from the group consisting C1, N2, T5, E6, R8, R9, HI 1, A12, R13, A14, S15, H16, L17, G18 and A20 has been substituted for the manufacture of an antimicrobial composition for the reduction and/or elimination of microorganisms or prophylactic treatment of a microbial infection.

2. Use according to claim 1, wherein the substitution(s) is/are selected from the group consisting of C1G₅ N2S, N2T, N2K, T5E, T5D, T5N, E6A, E6V, E6L, E6I, E6M, E6F, E6Y, E6W, R8A, R8V, R8L, R8I, R8M, R8W, R8K, R9K, H1 IA, H1 IV, H1 IL, H1 II, H1 IM, H1 IK₅ H1 IR, H1 IW, A12L, R13K, A14V, A14L, AI 41, A14M, S15A, S15V, S15L, S1 51, S15M, S15T, S15N, S15Q, S15K, S15R, S15W, H16K, H16R, H16A, H1 6V, H16L, H1 61, H16M, L17K, L17R, L17A, L 17V, L 171, L17M, G18W and A20R.

3. Use according to claim 2, wherein the substitution(s) is/are selected from the group consisting of, such as selected from the group consisting of N2S, N2K, T5E, T5D, T5N, E6A, E6V, E6L, E6I, E6M, E6F, E6Y, E6W, R8A, R8V, R8L, R8I, R8M, R8K, R8W, H1 IA, HI IV, H1 IL, H1 II, H1 IM, H1 IK, H1 IR, HI 1W, A12L, A14L, S15A, S15V, S15L, S 151, S15M, S15T, S15N, S15Q, S15K, S15R, S15W, H16K, H16R, H16A, H16V, H16L, H1 61, H16M, L17K, L17R, L17A, L17V, L 171, L17M, G1 8W and A20R.

4. Use according to claim 1, wherein the amino acid sequence of the peptide differs in 2, 3, 4, 5, 6, 7, 8, 9, 10, 11 or 12 amino acid residues from the amino acid sequence as shown in SEQ ID NO:1.

5. Use according to claim 1, wherein at least one amino acid residue selected from the group consisting of C1, N2, T5, E6, R8, R9, HI 1, A12, R13, A14, S15, H16, L17, G18 and A20 has been removed from the amino acid sequence shown in SEQ ID NO:1.

6. Use according to claim 5, wherein 1, 2, 3, 4, 5, 6, 7, 8, 9, 10 or 11 amino acid residues have been removed.

7. Use according to claim 1, wherein said peptide is linked to one or more amino acid residues, other peptide(s) or other substances.

8. (canceled)

9. Use according to claim 1, wherein the peptide is modified by amidation, esterification, acylation, acetylation, PEGylation or alkylation.

10. Use according to claim 1, wherein the antimicrobial composition is a pharmaceutical composition comprising a pharmaceutically acceptable buffer, diluent, carrier, adjuvant or excipient.

11. Use according to claim 10, wherein the composition comprises a salt.

12. Use according to claim 11, wherein the salt is selected from the group consisting monovalent sodium, potassium or divalent zinc, magnesium, copper or calcium.

13. Use according to claim 12, wherein the salt is divalent zinc.

14. Use according to claim 10, wherein the pharmaceutical composition has a pH from about 4.5 to about 7.0.

15. Use according to claim 1, wherein the antimicrobial or pharmaceutical composition comprises a mixture of between 1, 2, 3 or 4 different polypeptides as defined in claims 1-9.

16. Use according to claim 1, wherein the antimicrobial or pharmaceutical composition comprises one or more antibiotic and/or antiseptic agent(s).

17. Use according to claim 16, wherein the agent is selected from the group consisting of penicillins, cephalosporins, carbacephems, cephamycins, carbapenems, monobactams, aminoglycosides, glycopeptides, quinolones, tetracyclines, mac-rolides, fluoroquinolones, iodine, silver, copper, clorhexidine, polyhexanide, biguanides, chitosan, acetic acid, and hydrogen peroxide.

18. Use according to claim 1, wherein the antimicrobial or pharmaceutical composition is in the form of granules, powders, tablets, coated tablets, capsules, suppositories, syrups, injectable forms, emulsions, gels, ointments, suspensions, creams, aerosols, droplets.

19. Use of a polypeptide which shows at least 70% homology to SEQ ID NO:2 for the manufacture of an antimicrobial composition for the reduction and/or elimination of microorganisms.

20. Use of the polypeptide according to claim 19, wherein the polypeptides show 80%, 90% or 95% homology to SEQ ID NO:2 for the manufacture of an antimicrobial composition for the reduction and/or elimination of microorganisms.

21. Use according to claim 1 wherein the microorganism is selected from the group consisting of bacteria, virus, parasites, fungus and yeast.

22. Use according to claim 21, wherein the microorganism is selected from the group consisting of Gram positive and Gram-negative bacteria such as Enterococcus faecalis, Escherichia coli, Pseudomonas aeruginosa, Proteus mirabilis, Streptococcus pneumoniae, Streptococcus pyogenes, Staphylococcus aureus, virus, parasites, fungus and yeast, such Candida albicans and Candida parapsilosis.

23. Use according to claim 1, wherein the antimicrobial or pharmaceutical composition is comprised in bandages, plasters, sutures, soap, tampons, diapers, shampoos, tooth paste, anti-acne compounds, suncreams, textiles, adhesives, incorporated in wound dressings, cleaning solutions or implants.

24. Use according to claim 1, wherein the antimicrobial composition is used for the treatment of a disease selected from the group consisting of prophylactic treatment of burn wounds, after surgery and after skin trauma, atopic dermatitis, impetigo, chronic skin ulcers, infected acute wound and burn wounds, acne, external otitis, fungal infections, pneumonia, seborrhoeic dermatitis, candidal intertrigo, candidal vaginitis, oropharyngeal candidacies, eye infections and nasal infections.

25. A method of treating a mammal having a microbial infection, comprising administering to a patient a therapeutically effective amount of an pharmaceutical composition according to claim 10.

26. A method of treating a mammal according to claim 25, wherein the mammal is selected from the group consisting of humans, horses, dogs, cats, cows, pigs and camels.


SEQUENCE LISTING (2 sequences)

SEQ ID NO: 1 - protein, 21 aa, human
CNYITELRRQHARASHLGLAR

SEQ ID NO: 2 - protein, 77 aa, human
SVQLTEKRMDKVGKYPKELRKCCEDGMRENPMRFSCQRRTRFISLGEACKKVFLDCCNYITELRRQHARASHLGLAR